(19) United States
(12) Patent Application Publication
(10) Pub. No.: US 2014/0120069 A1
(43) Pub. Date: May 1, 2014

(54) Amphoteric Materials Based on Crosslinked Hyaluronic Acid, Method of Preparation Thereof, Materials Containing Entrapped Active Agents, Method of Preparation Thereof, and Use of Said Materials

(71) Applicant: CONTIPRO BIOTECH S.R.O., Dolni Dobrouc (CZ)

(72) Inventors: Gloria Huerta-Angeles, Ceska Trebova (CZ); Drahomira Chladkova, Letohrad (CZ); Radovan Buffa, Humenne (SK); Sofiane Kettou, Uherske Hradiste (CZ); Vladimir Velebny, Zamberk (CZ)

(73) Assignee: CONTIPRO BIOTECH S.R.O., Dolni Dobrouc (CZ)

(21) Appl. No.: 14/113,527

(22) PCT Filed: Apr. 19, 2012
(86) PCT No.: PCT/CZ2012/000035

§ 371 Date: Jan. 8, 2014

(30) Foreign Application Priority Data
Apr. 24, 2011 (CZ) PV 2011-241

(51) Int. Cl. A61K 47/36; A61K 31/704; A61K 35/32; A61K 31/416

(52) U.S. Cl. CPC A61K 47/36; A61K 31/416; A61K 31/704; A61K 35/32
USPC 424/93.7; 536/28.7; 514/777; 514/407; 514/34

(57) ABSTRACT

The present invention describes novel amphoteric materials based on crosslinked hyaluronic acid, according to the general formula (I), and a method of preparation of said materials. Further, the invention relates to the material containing entrapped active agents (e.g. drugs, growth factors etc.) and a method of preparation thereof. Moreover, the present invention relates to the use of said materials for controlled release systems, in tissue engineering, wound dressing or tissue regeneration.

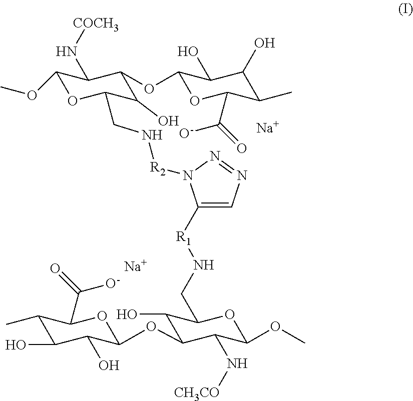

### FIELD OF THE INVENTION

The present invention describes novel amphoteric materials based on crosslinked hyaluronic acid, and a method of preparation of said materials. Further, the invention relates to the material containing entrapped active agents (e.g. drugs, growth factors etc.) and a method of preparation thereof. Moreover, the present invention relates to the use of said materials for controlled release systems, in tissue engineering, wound dressing or tissue regeneration.

### STATE OF THE ART

The present invention relates to a biocompatible material or hydrogel formed from a chemically modified polyanionic polysaccharide, to be specific hyaluronic acid. Hyaluronic acid or hyaluronan (HA) is a naturally occurring mucopolysaccharide consisting of alternating D-glucoronic acid and N-acetyl-D-glucosamine joined together by alternating beta 1-3-glucoronic and beta 1-4-glucosamine bonds into a lineal polymer of high molecular weight. HA occurs in extracellular matrix and plays a vital role in maintaining tissue integrity. HA facilitates adhesion and differentiation of cells during inflammation, wound repair, and embryonic development. In animal models, topically applied HA accelerated dermal wound healing and decreased fibrosis and scar formation in rats and hamsters. HA is used as a carrier for wound healing agents, in cosmetic formulations and drug delivery because it presents high water retention capacity and good biodegradability. HA is associated with a variety of biological processes, such as tumorigenesis, morphogenesis, inflammation, and host response to injury.

The native hyaluronic acid has a major drawback: it degrades rapidly. Hyaluronan degradation modifies its viscoelastic and mechanical properties and that limits the above mentioned applications. In recent years there has been an increasing interest in chemically modified derivatives of HA which would be less sensitive towards degradation, enzymatic attack and temperature changes. HA in a chemically modified form is useful as a surgical aid and prevent adhesions of body tissues during post-operation period.

The present invention describes the design and synthesis of new amphoteric derivatives of hyaluronic acid for the preparation of hydrogels that meet requirements such as biodegradability and biocompatibility needed for biomedical applications, including controlled release (Pal, Paulson, Rousseau, Stefan, Ian & Johan, 2009) tissue engineering and wound dressing. The super porous insoluble derivates of hyaluronic acid have different transport system (diffusion), Fickian, anomalous and super case II transport as response to the change of the environmental pH.

pH-responsive hydrogels are a kind of materials that can react to changes in the environmental pH and alter their volume accordingly. They are especially suitable for controlled drug delivery system (CDDS) since there are pH variations at several body sites, including the gastrointestinal tract, vagina and blood vessels (Gupta, Vermani & Garg, 2002). In recent years, particular interests have been devoted to amphoteric pH-sensitive hydrogels that posses both positive and negative charges and therefore are capable of swelling in both acidic and basic medium (Yao, Chen, Liu & De Yao, 2003). In this invention, the swelling-shrinking switch had allowed a constant release rate which is adequate for a sustained release dosage matrix. The process for the preparation of the devices according to the invention consists in the chemical modification of sodium hyaluronate (or hyaluronan) by a process of oxidation-reductive amination in order to introduce secondary amines bearing azido or alkyne moieties borne by the linear polymeric backbone, which are able to crosslink via click chemistry. Those chemically stable secondary amines of sodium hyaluronate reported in this invention are partially modified-primary alcohols in the glucuronic acid of hyaluronic acid.

The current necessity of targeting specific cells and organs requires the location of the delivery to be very specific. Burst and a prolonged release are necessary in several pulsatile release processes, so that the active ingredient can be delivered rapidly and efficiently upon changes of environmental conditions which trigger the release. Most of the published work had reported formulations which cannot release the active ingredient completely.

The reaction conditions described before had chemically modified the primary alcohols of the polysaccharide, which means that the reaction is not chemoselective. Crescenzi et al. have described the use of the “click chemistry” method for the obtention of hydrogels based on hyaluronan (Crescenzi, Cornelio, Di Meo, Nardecchia & Lamanna, 2007; Testa et al., 2009). The authors described the chemical modification of hyaluronic acid carried out by activation of the carboxylic groups by 1-ethyl-3-(3-dimethylaminopropyl) carbodiimide (EDCI).

As is known in literature, one of the main drawbacks of the carbodiimide activation is the formation of N-Acyl-N—N′di-substituted urea as a reaction by-product. EDCI degrade fast in water and there is necessity of using an excess of the reagent for obtaining chemical modification, which increases the cost of the process. The reaction conditions cannot be optimalized using EDCI in order to increase degree of substitution. Crescenzi et al have produced hydrogels which have not presented such very well organized structure. Organized walls and porosity are important for applications. An interconnected network that resembles natural structures is characterized by fast diffusion and high biocompatibility, Crescenzi's material lack this property. The main drawback of Crescenzi's et al is their reduced release efficiency. It is known that an incomplete release decreases the bioavailability of the therapeutic agent and alters the overall delivery profile. After the initial burst, the authors claim that most heavily crosslinked hydrogels should guarantee a prolonged release but the authors have not shown experimental evidence for this affirmation; however, the crosslinking degree was not drastically increased in Crescenzi's work.

On the other hand, materials described on patent WO-2008/03525 reported a maximum quantity of benzydamine hydrochloride released over a period of about 3.5 hours and equal to 88%. However, the materials described in the present patent application, using amphoteric derivates are able to retain in 8 hours a similar amount of benzydamine hydrochloride. Therefore, the hydrogel according to this invention has a matrix structure which allows a prolonged and controlled release.

Different reports of amphoteric hydrogels found in literature involved synthetic hydrogels mainly composed of poly (acrylic acid) and its derivates (Luo, Peng, Wu, Sun & Wang). Those materials are not biodegradable. Natural amphoteric hydrogels, such as chitosan-based ones crosslinked with glutaraldehyde are biodegradable and biocompatible. However, compositions are uncontrollable and properties are often varied from batch to batch (El-Sherbiny & Smyth; Chen, Tian & Du, 2004; Shang, Shao & Chen, 2008). Hybrid hydrogels containing both synthetic and natural polymers have been proposed attempting to combine the merits of synthetic and natural polymers. However, they are not completely biodegradable (Ferruti, Bianchi, Ranucci, Chiellini & Caruso, 2005).

Different materials have been produced using click chemistry and patented as well, for example WO 2007/035296 discloses hydrogels which are formed from hydrophilic polymers that are crosslinked with bi- or multifunctional linkers which are cycloaddition reactive. Similarly (Malkoch et al., 2006) presented hydrogels based on polyethylene glycol which are synthesized by click chemistry using tetrafunctional azide crosslinkers. Potential disadvantages of these hydrogels include their reliance on small bi- or multifunctional, highly reactive, crosslinking agents of unknown biocompatibility, which would limit the use of these hydrogels e.g. for in-situ applications in which the hydrogel is prepared from reactants that will be in contact with the living tissue. Crosslinking through “click chemistry” was also applied by DuPrez et al in the patent application EP 2090592-A1. Therein, dextran is modified using carbonyldiimidazole activation. Dextran-azidopropylcarbonate and dextran-propargylcarbonate derivatives can be consequently crosslinked into chemical hydrogels. The authors predicted that the click-linked carbonate esters can also hydrolyze under physiological conditions; however the patent has not shown any biological compatibility, cytotoxicity and biodegradability. Other authors had used click chemistry on polysaccharides, as a general approach to develop (1-->3)-[beta]-D-glucans with various functional appendages (Hasegawa et al., 2006).

Tankam et al had demonstrated that starch propargyl ethers are valuable intermediates for the preparation of functional polysaccharides for their use in a 1,3-dipolar cycloaddition with benzyl azide (‘click-chemistry’) to create an N-benzyltriazole derivatized starch. (Tankam, Müller, Mischnick & Hopf, 2007).

### SUMMARY OF THE INVENTION

Therefore, the object of the invention is to find hydrogels which exhibit more advantages of the before known materials, and especially overcome the aforementioned disadvantages. More specifically, the problem to be solved by the invention is to obtain a non-cytotoxic, biocompatible material based on HA, having an improved drug release characteristics. This problem is solved by the amphoteric material according to the invention which has a much more organized porosity, interconnected pores, a higher absorption capacity and moisturizing capacity, constant drug release rate and higher release of the entrapped drug. Further objects will become obvious on the basis of the following description and claims.

The drawbacks of the prior art are overcome and the problem is solved by the crosslinked derivative of hyaluronic acid of the invention, according to the formula I:

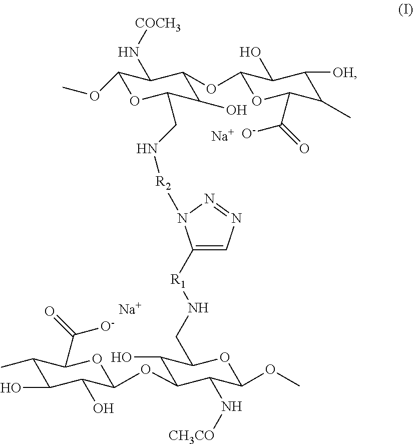

wherein R₁ and R₂ are independently the same or different and is an aliphatic, aromatic, arylaliphatic and cycloaliphatic moiety, optionally containing a heteroatom O, which contains 3-12 carbons. A non-limiting examples of R₁ is methyl and a non-limiting example of R₂ is propyl.

Further, the invention relates to a process of preparation of said derivative, comprising the steps of:

i) preparation of a secondary amine hyaluronan derivative carrying an alkynyl group, of the formula II:

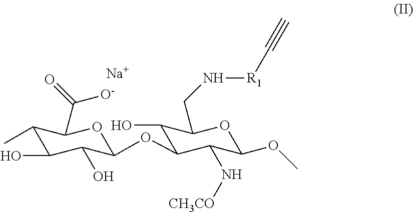

ii) preparation of a secondary amine hyaluronan derivative carrying an azidyl group, of the formula III:

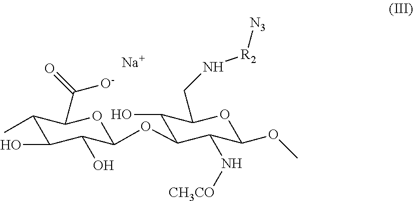

iii) mixing the derivative of the formula (II) and the derivative of the formula (III), and
iv) cycloaddition reaction of the derivative of the formula (II) and the derivative of the formula (III) in the presence of CuSO₄ and sodium ascorbate to obtain the crosslinked derivative of the hyaluronic acid.

Step i) preferably comprises the steps of a) a chemoselective oxidation of hyaluronan in the C-6 position, b) coupling a primary amine carrying a terminal alkynyl group to the oxidized hyaluronan to form alkynyl-imine hyaluronan, c) reduction of the alkynyl-imine hyaluronan to form a secondary alkynyl-amine hyaluronan; wherein the step a) may be followed by isolation of the oxidized hyaluronan or all of the steps a) to c) are performed in one pot.

Similarly, step ii) preferably comprises the steps of a) chemoselective oxidation of hyaluronan in the C-6 position, b) coupling a primary amine carrying a terminal azidyl group to the oxidized hyaluronan to form azidoalkyl-imine hyaluronan, c) reduction of the azidoalkyl-imine hyaluronan to form a secondary azidoalkyl amine hyaluronan; wherein the step a) may be followed by isolation of the oxidized hyaluronan or all of the steps a) to c) are performed in one pot.

The primary amine carrying a terminal alkynyl group can be for example propargyl amine or ethynyl aniline and the primary amine carrying a terminal azidyl group can be for example 3-azidopropan-amine, 11-azido-3,6,9-trioxaundecan-1-amine or azido-aniline.

The oxidation agent used in step a) of either of steps i) and ii) may be the system 2,2,6,6,-tetramethylpiperidine-1-oxyl radical (TEMPO)/sodium hypochlorite (NaClO) in the presence of NaBr or NaCl, or Dess-Martin Periodinane (DMP).

The crosslinked derivative obtained in step iv) can be in the form of a gel which is then freeze-dried, preferably by liquid nitrogen or by ice.

Step iii) may further involve adding biologically active substances into the reaction mixture, said substances being selected from the group comprising drugs, proteins, enzymes, biopolymers and biologically compatible synthetic polymers. The drugs can be selected from the group comprising e.g. analgesics, antibiotics, antimicrobial, cytostatics, anticancer, anti-inflammatory, wound healing agents and anesthetics.

Further, step iv) can be followed by seeding the formed crosslinked derivative with growth factors such as chondrocytes.

The crosslinked derivative of hyaluronic acid of the formula (I) can be e.g. in the form of a gel or a scaffold and may further comprise entrapped biologically active substances selected from the group comprising drugs, proteins, growth factors, enzymes, biopolymers and biologically compatible synthetic polymers.

Moreover, the invention further relates to the use of the crosslinked derivative for controlled release systems, in tissue engineering, wound dressing or tissue regeneration.

### BRIEF DESCRIPTION OF THE DRAWINGS

FIG. 1 depicts the chemical representation of the crosslinked derivative of hyaluronic acid of the invention, according to the formula I, and the amphoteric network thereof.

FIG. 2 shows ¹H-NMR spectrum of propargyl 2-amine-hyaluronan (HA-CAPr) in D₂O (500 MHz).

FIG. 3 shows ¹H-NMR spectrum of azidopropyl-2-amine-hyaluronan (HA-CAPA) in D₂O (500 MHz).

FIG. 4 shows the infrared spectra (thin film) of crosslinked material.

FIG. 5 represents water uptake for hydrogel I in solutions of different ionic strength (1M NaCl, 0.1M NaCl, 0.01M NaCl, 0.001M NaCl and 0.0001M NaCl). The curves represented higher swelling degree with the increasing of the ionic strength of the media.

FIG. 6 shows a photo of water uptake as a function of ionic strength; from left to right: 1M NaCl, 0.1 M NaCl, 0.01 M NaCl, 0.001 M NaCl and 0.0001 M NaCl.

FIG. 7 represents the kinetics of swelling of the crosslinked material (I) as a function of pH.

FIG. 8 represents the kinetics of swelling of the crosslinked material (I) as a function of the ionic strength.

FIG. 9 represents cumulative release of benzydamine in water.

FIG. 10 represents cumulative release of benzydamine in PBS.

FIG. 11 represents cumulative release of benzydamine in buffer pH=6.0.

FIG. 12 depicts the cumulative release of Doxorubicin in water.

FIG. 13 shows the availability of derivative propargyl 2-amine-hyaluronan tested on 3T3 cells in time.

FIG. 14 shows the availability of derivative azidopropyl-2-amine hyaluronan tested on 3T3 fibroblasts in time.

FIG. 15 shows the cytotoxicity of crosslinked material as obtained in example 9 tested on 3T3 cells in time.

FIG. 16a-b represents a live-dead cell analysis of chondrocytes seeded into scaffold built from click-chemistry based hydrogel. The microscope images are taken of the surface of the scaffold on the 6^{th} day of the cultivation or when cultivation had started (FIG. 16a) and on the 15^{th} day of the cultivation. FIGS. 16c-d show microscope images of the central part of the scaffold: on the 6^{th} day of the cultivation (FIG. 16c) and on the 15^{th} day of the cultivation (FIG. 16d).

FIG. 17 shows bioavailability of the material tested on chondrocytes as a function of time. Chondrocytes were cultivated for 21 days.

FIG. 18 represents a surface image of the material as well as a transversal cut of the material in the swollen state after freeze-drying by liquid N₂. This material was obtained using chemically modified HA of 200 kDa.

FIG. 19 represents a surface image of the material, as well as a transversal cut of the material in the swollen state after freeze-drying by ice. This material was obtained using chemically modified HA of 100 kDa.

FIG. 20 shows a SEM micropicture of the gel (transversal cut) in swollen state after freeze-drying. The hydrogel was incubated at 25° C. for 30 days in buffer pH=9.0.

FIG. 21 represents a SEM micropicture of the hydrogel obtained using low-molecular weight modified HA (18 kDa), freeze-dried in liquid N₂ (left) or ice (right).

### DETAILED DESCRIPTION OF THE INVENTION

In this description, certain expressions are used rather frequently as they relate to important technical aspects of features or embodiments. For some of these expressions, the following definitions should be used unless the specific context in which they are used requires a different interpretation.

Hyaluronic acid, as in the scope of the present patent application, refers to both the polysaccharide in its form of a polycarboxylic acid and its salts, such as sodium, potassium, magnesium and calcium salt. The hyaluronic acid used in the present invention can derive from any source; it can be obtained for example by extraction from chicken combs (EP 138572 B1), or by fermentation (EP 716688 B1), and can have a weight average molecular weight ranging from 50,000 to 3,000,000 Da.
Degree of substitution: in case of a polysaccharide backbone, a normally useful degree of substitution (DS) is defined as the reactive moieties per 100 saccharide dimers in this case hyaluronic acid, i.e. representing the number of dimers which were chemically modified.
Crosslinked material, as used herein, is a three-dimensional polymeric network made by chemical crosslinking of one or more hydrophilic polymers. This derivate is able to swell but does not dissolve in contact with water.
Linker, as used herein, is a bifunctional chemical structure (molecule or moiety) which connects two or more polymers covalently or non-covalently. This patent application describes the addition of aliphatic or aromatic amines containing the synthon A-R—X where A=NH₂, NH and R=aliphatic, aromatic, arylaliphatic, cycloaliphatic and heterocyclic substitutes containing from 3 to 12 carbons. X refers a group which is able to undergo a cycloaddition or sigmatropic reaction.
Selective oxidation is a process of chemoselective oxidation in the position C-6 on the hyaluronan backbone for the obtention of a geminal diol or aldehyde moiety borne by the biopolymer.
Oxidation and reductive amination in one pot-procedure is a process of conversion a primary alcohol borne by a polysaccharide in aldehyde or geminal diol and addition of a primary or secondary amino linker and reduction of the imine-intermediate without isolation of any component of the reaction in order to obtain a secondary or tertiary amine attached to the polymeric backbone.
Cycloaddition reaction: According to the invention a polymer has a moiety or functional group which is capable of undergoing a cycloaddition reaction. The cycloaddition reactions that are of particular interest are those that have recently received much attention in the concept of the so-called “click chemistry”, to be specific the so-called Huisgen-reaction (also called the Sharpless “click” reaction) (Huisgen, R., 1963). The azide/alkyne click reaction is a recent rediscovery of a reaction fulfilling many requirements for the affixation of linkers into polymers by post-modification process, which include's a) quantitative yields; b) high tolerance of functional groups and insensitivity of the reaction to solvents. The basic reaction described in this work, which is nowadays summed up under the name Sharpless-type click reaction, is a variant of the Huisgen 1,3 dipolar cycloaddition reaction between C—C triple, C—N triple, and alkyl-/aryl- or sulfonyl azides developed by Rostovtsev and simultaneously by Tornoe in 2002 (Rostovtsev, Green, Fokin & Sharpless, 2002; Tornøe , Christensen & Meldal, 2002).
Mechanism of drug release: The mechanism of drug release from swellable matrices is determined by several physical-chemical phenomena. Among them, polymer water uptake, gel layer formation and polymeric chain relaxation are regarded as primarily involved in the modulation of drug release. Eq. (1) is currently used for the analysis of drug release process in order to categorize the predominant mechanism (Korsmeyer et al., 1983).

Mt/M∞=ktⁿ Eq. (1)

Mt/M∞ ratio is the proportion of drug released at time t, k is the kinetic constant, and the exponent n has been proposed as indicative of the release mechanism. In this context, n=0.5 indicates Fickian release (diffusional controlled release) and n=1 indicates a purely relaxation controlled delivery which is referred as Case II transport. Intermediate values indicate an anomalous behavior (non-Fickian kinetics corresponding to coupled diffusion/polymer relaxation) (Ritger & Peppas, 1987a). Occasionally, values of n>1 have been observed, which has been regarded as Super Case II kinetics.
Sustained release dosage form is a medical device characterized by drug release at a predetermined rate or by maintaining a constant drug level for a specific period of time. It provides an amount of drug initially made available to the body to cause the desired therapeutic response, followed by a constant release of the medication for maintenance of activity over a period of time. Sustained-release implies slow release of the drug over a time period; it may or doesn't have to be a controlled-release. Sustained release means that the drug will be released under first order kinetics, or independent of reaction parameters.
Controlled release dosage form: is a perfectly zero-order release; thereafter, the drug releases over time irrespective of the concentration thereof.
Amphoteric materials (AM): active materials that contain or have the property to form both positive and negative charged groups under determined environmental conditions. They are able to impart special properties to a formulation such as sustained release and special swelling properties. Their nature can make them especially useful in applications requiring biological contact. Most studied amphoteric hydrogels fall into one of the three categories: synthetic, natural and hybrid ones.
Molecular weight cut-off is the phenomena attributed to a hindered diffusion as the guests negotiated a more torturous diffusional path through the material, which means that the material possesses a network-intricate structure that may retain inside small molecular weight components.

In a first aspect, the invention relates to the crosslinked derivative of hyaluronic acid according to the formula I:

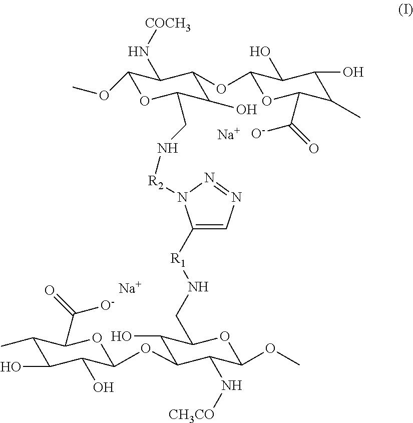

wherein R₁ and R₂ are independently the same or different and are an aliphatic, aromatic, arylaliphatic, cycloaliphatic and heterocyclic moiety containing from 3-12 carbons.

The present invention provides new kinds of materials that are characterized as chemically stable, highly porous, non cytotoxic and biocompatible, and which can be used for different applications known by those skilled in the art. Characterization of the materials has provided evidence of a high connectivity and diffusion that is the clue for the sustained and controlled release of substances. This patent application describes materials where bioactive biologically or pharmacologically active molecules or macromolecules can be physically incorporated before or after crosslinking. Those kinds of hydrogels had presented sustained release upon changes of the biological microenvironment. The application of these materials may be particularly advantageous for wound treatment where an initial burst release followed by a diminishing need for a drug is necessary. The materials reported in this patent application are able to establish specific acido-base interactions which give them special advantages.

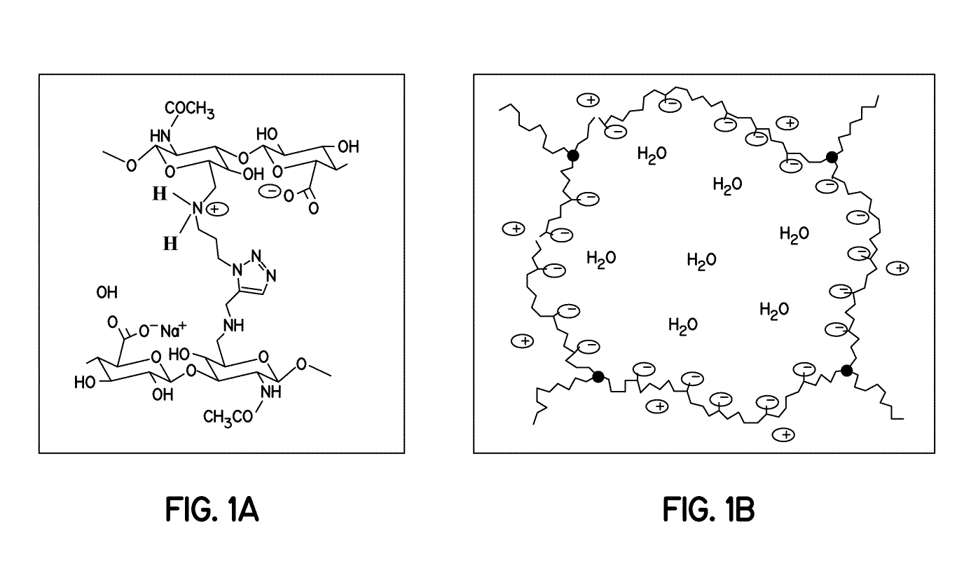

In a second aspect, the invention concerns a process for making a water insoluble amphoteric material based on chemically modified HA (FIG. 1); this process includes chemical modification of hyaluronic acid by oxidation and reductive amination in two steps and reaction depicted in Schemes: 1, 2, 4 and 5, or one pot (Scheme 3 and 6) below. The process results in amphoteric derivatives of a polyanionic polysaccharide, wherein at least one of the polysaccharide chains consists of hyaluronic acid, crosslinked via 1,3dipolar cycloaddition. The linkers are attached covalently via secondary amine to the anionic polysaccharide (FIG. 1), which possesses a microporous morphology and tailored microstructure.

More specifically, the process according to the invention comprises the steps of i) preparation of a secondary amine hyaluronan derivative carrying an alkynyl group; ii) preparation of a secondary amine hyaluronan derivative carrying an azidyl group; iii) mixing the derivative of step i) and the derivative of step ii); iv) and cycloaddition reaction of the mixed derivatives in the presence of a catalyst to obtain a crosslinked derivative of the hyaluronic acid.

The preparation of the secondary amine hyaluronan derivative carrying an alkynyl group (step i)) named as well dipolarophiles, alkenes and alkynes and molecules that possess related heteroatom functional groups, according to the formula II:

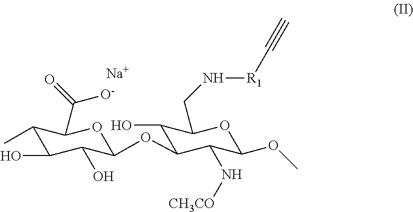

may be carried out in two steps, isolating the oxidized intermediate before the reductive amination takes place, or in one pot. The two-step preparation corresponds to the scheme 1 or 2 (depending on the type of the oxidant used):

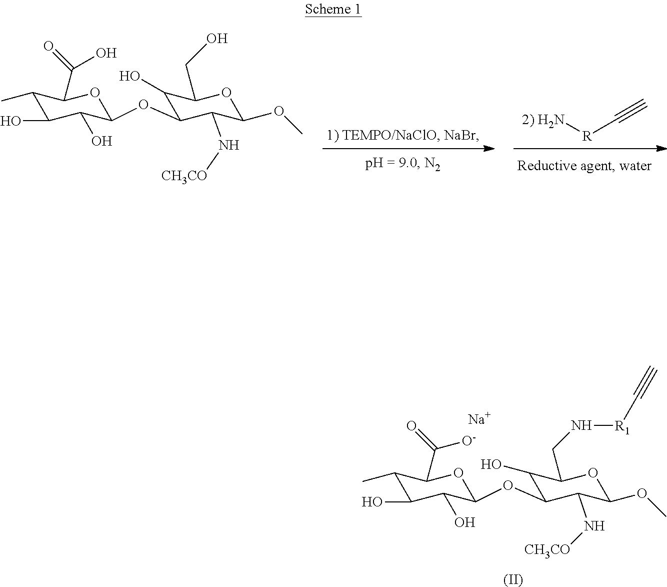

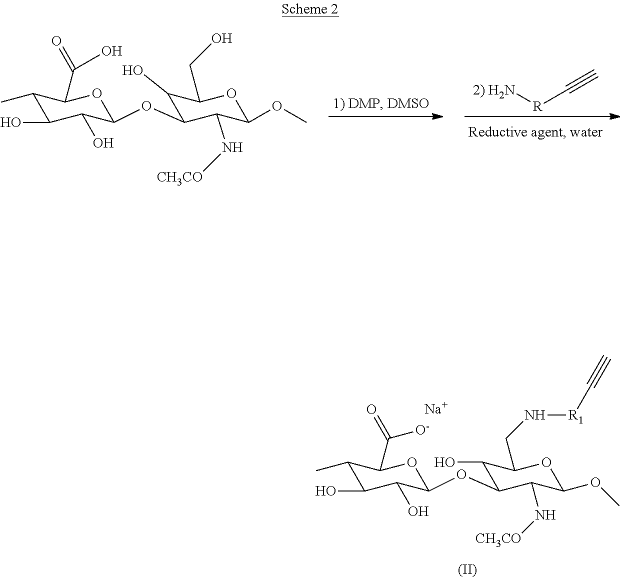

comprising a) chemoselective oxidation of hyaluronan in the C-6 position, b) coupling a primary amine carrying a terminal alkynyl group to the oxidized hyaluronan to form alkynyl-imine hyaluronan, c) reduction of the alkynyl-imine hyaluronan to form a secondary alkynyl-amine hyaluronan. Scheme 1 shows oxidation reaction carried out using TEMPO, Sodium Bromide, and NaClO in water or buffer for the selective oxidation of C-6. The reaction product was isolated and purified. The second step consists of the addition of a primary amine carrying an alkynyl group to form an imine which is reduced to a secondary amine. Scheme 2 shows oxidation reaction carried out using Dess-Martin Periodinane in DMSO for the selective oxidation of C-6. The reaction product was isolated and purified. The second step consists of the addition of a primary amine bearing an alkynyl group in water to form an imine which is reduced to a secondary amine.

In case of the one-pot preparation where no isolation is necessary, the reaction scheme is depicted below:

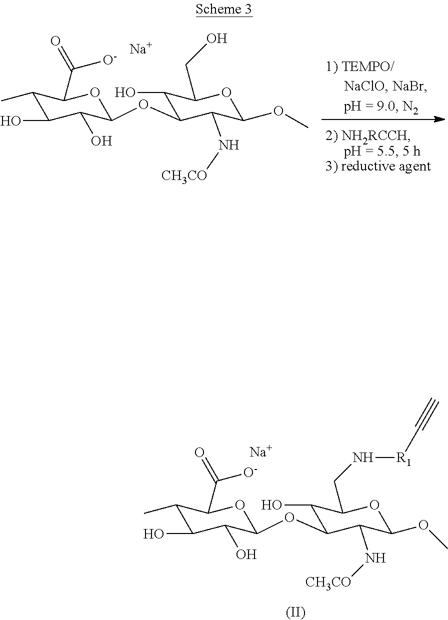

Scheme 3 shows oxidation and reductive amination procedure carried out in one pot for the selective modification of C-6 bearing an alkynyl group named II.

The following step ii), i.e. the preparation of a secondary amine hyaluronan derivative carrying an azidyl group or 1,3-dipolar compounds which may contain one or more heteroatoms and can be described as having at least one mesomeric structure that represents a charged dipole and represented by the formula III:

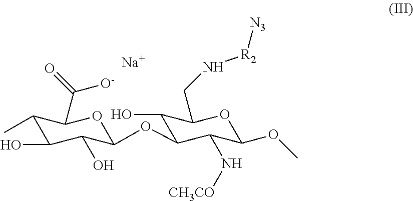

may be again carried out in two steps, isolating the oxidized intermediate before the reductive amination takes place, or in one pot. The two-step preparation corresponds to the scheme 4 or 5 (depending on the type of the oxidant used):

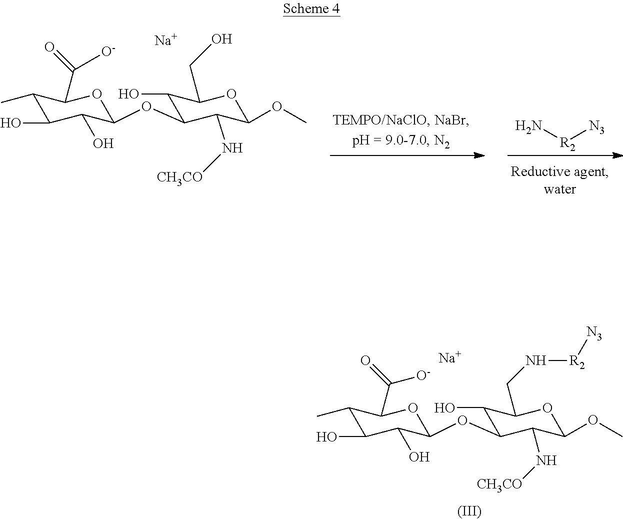

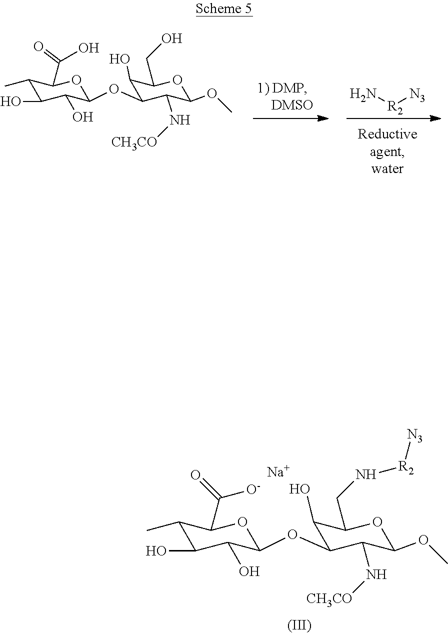

comprising the steps of a) chemoselective oxidation of hyaluronan in the C-6 position, b) coupling a primary amine carrying a terminal azidyl group to the oxidized hyaluronan to form azidoalkyl-imine hyaluronan, c) reduction of the azidoalkyl-imine hyaluronan to form a secondary azidoalkyl amine hyaluronan. Scheme 4 shows oxidation reaction carried out using TEMPO, Sodium Bromide, and NaClO in water or buffer for the selective oxidation of C-6. The reaction product was isolated and purified. The second step consists of the addition of a primary amine carrying an azidyl group to form an imine which is reduced to a secondary amine or derivative named III. Scheme 5 shows oxidation reaction carried out using Dess-Martin Periodinane in DMSO for the selective oxidation of C-6. The reaction product was isolated and purified. The second step consists of the addition of a primary amine bearing an azidyl group in water to form an imine which is reduced to a secondary amine or derivative (III).

In case of the one-pot preparation where no isolation is necessary, the reaction scheme is depicted below:

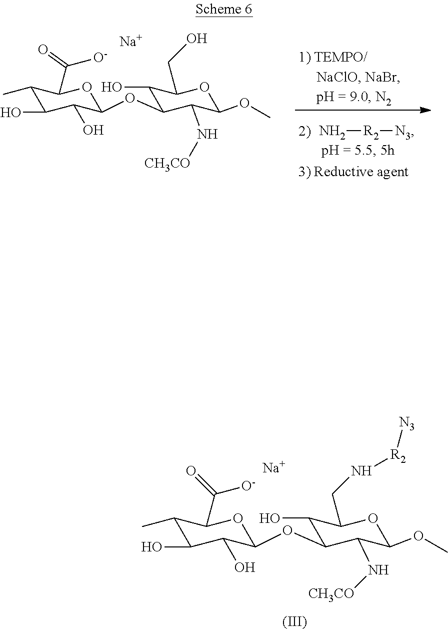

Scheme 6 shows oxidation and reductive amination reaction carried out in one pot for the selective modification of C-6 bearing an azidyl group (III).

The process described in schemes 3 and 6 is preferably carried out in the following way: the hyaluronic acid is reacted with an oxidant, for 15 minutes at a pH from 9 to 12, quenched by changing pH from 5-8, or adding a primary alcohol, and then reacted with a primary amine bearing an azidyl or alkynyl moiety, which have produced chemically modified derivates of hyaluronic acid with a mean molecular weight of 100-200 kDa. The cycloaddition reaction of the derivative of the formula (II) and the derivative of the formula (III) in the presence of CuSO₄ and sodium ascorbate results in the crosslinked derivative of the hyaluronic acid, according to the reaction scheme 7:

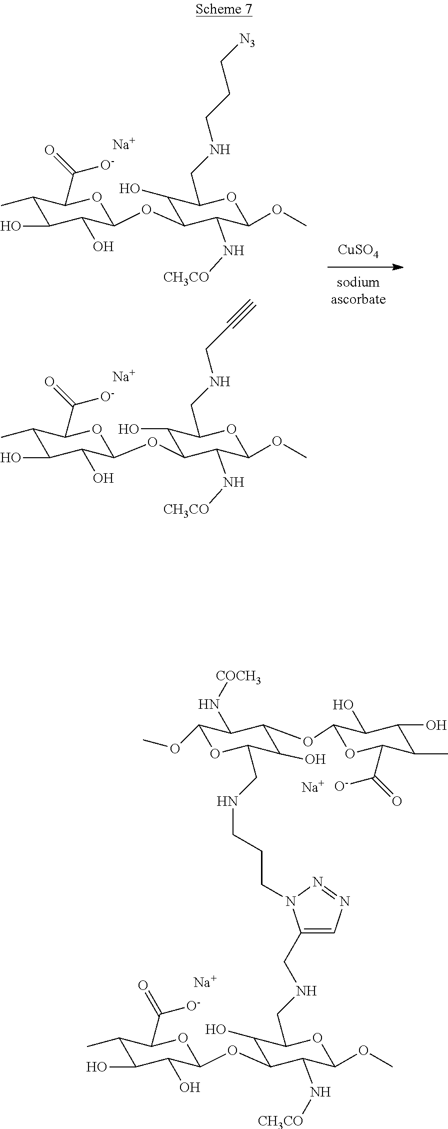

Scheme 7 represents the cycloaddition reaction of derivates propargyl-2-amine hyaluronan (II) and of azidopropyl-2-amine hyaluronan (III).

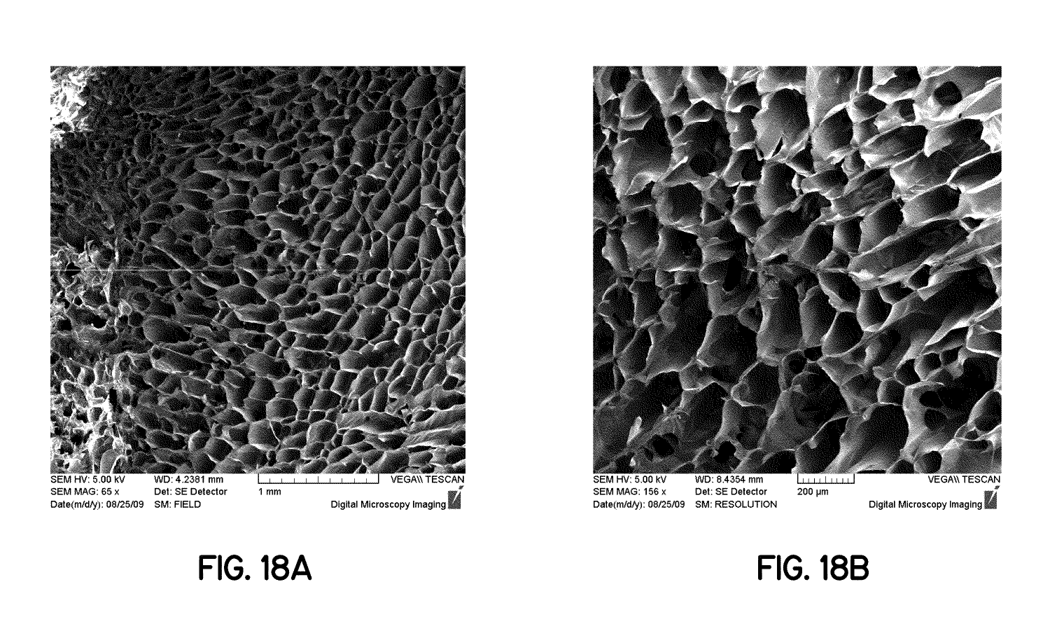

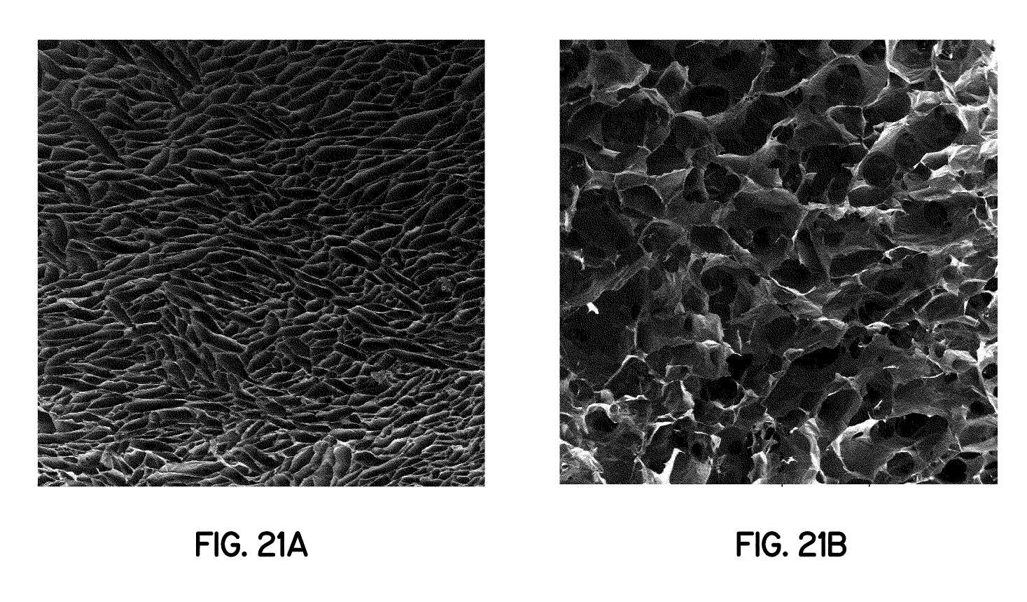

Macroporous materials obtained using the cycloaddition reaction of Scheme 7 is depicted in FIGS. 18-21. The images obtained by scanning electronic microscope (SEM) revealed that materials posses highly organized structures.

The first step or oxidation may use different oxidant agents, such as the system 2,2,6,6,-tetramethylpiperidine-1-oxyl radical (TEMPO)/Sodium hypochlorite (NaClO) and using as an additive NaBr or NaCl, as shown in Schemes 1, 3, 4 and 6, or Dess-Martin Periodinane (DMP) as depicted in schemes 2 and 5. The oxidation chemoselectivity modified the C-6 on the hyaluronan backbone. The formed geminal diols or “aldehydes” can react with different primary amines, aliphatic or aromatics bearing terminal alkyne or azide groups. First, a moiety carrying an alkynyl group, such as a propargyl substitute, is introduced as described in Scheme 1, 2 and 3 by a sequence of a chemoselective oxidation in C-6 as a process of oxidation-isolation-reductive amination (Schemes 1 and 2) or oxidation-reductive amination in one step procedure (Scheme 3).

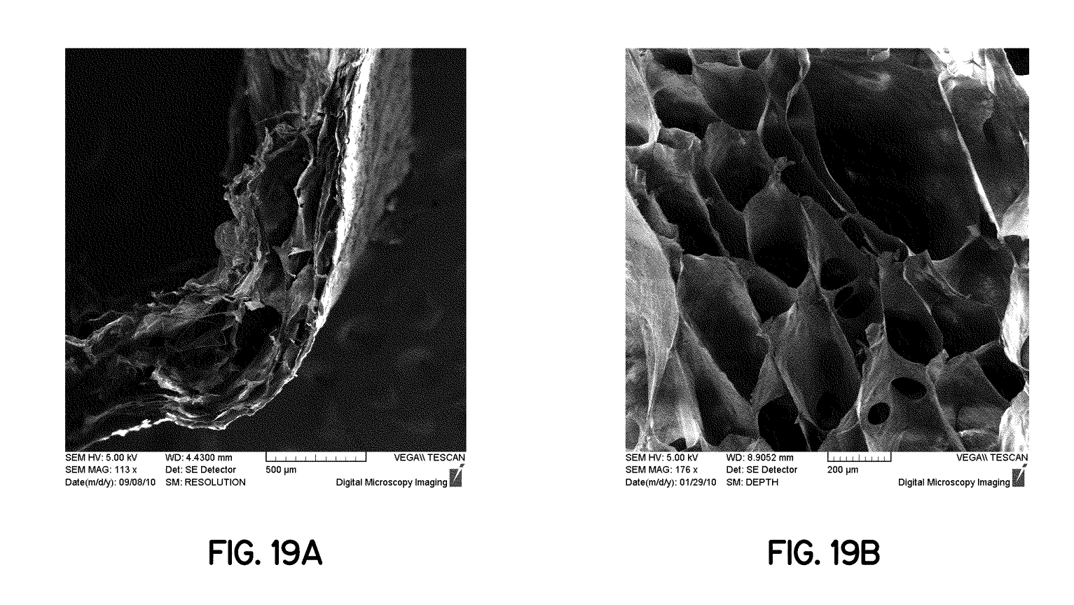

The oxidation reaction using NaClO/TEMPO/NaBr system in one step procedure was carried out on phosphate or carbonate buffers, using a controlled buffered pH or water from 5° C. to 25, preferably at 5° C. under nitrogen atmosphere, which allowed a lower degradation of the modified polymer. The reaction was quenched by changing pH or adding IPA or ethanol in order to deactivate the oxidant agent. The oxidation using DMP was carried out in DMSO. The process of oxidation is carried out using native hyaluronic acid, possessing different molecular weights (from 20 kDa to 2000 kDa). The complete process of oxidation and reductive amination produces derivates from 18 to 1000 kDa, preferably 18 to 200 kDa which produce the most stable hydrogels (FIG. 19).

Secondly, the secondary amine hyaluronan derivative carrying an alkynyl group, e.g. 3-azidyl propyl-2-amine-hyaluronic acid was prepared as described in Scheme 4, 5 and 6 by a sequence of a chemoselective oxidation in C-6 and reductive amination in one step (Scheme 6) or in oxidation-isolation-reductive amination procedure (Schemes 4 and 5). The oxidation reaction was carried out in phosphate or carbonates buffer, using a control tampon system pH or in water from 5° C. to 25, preferably at 5° C. under nitrogen atmosphere. The derivate obtained by DMP oxidation also undergoes the addition of primary amines in water, or phosphates buffer. DMP oxidation degrade considerably hyaluronic acid, however the produced hydrogels are stable.

Said chemically modified hyaluronic acid derivatives (II and III) have a molecular weight ranging from 2 to 1000 kDa, preferably from 18 to 200 kDa.

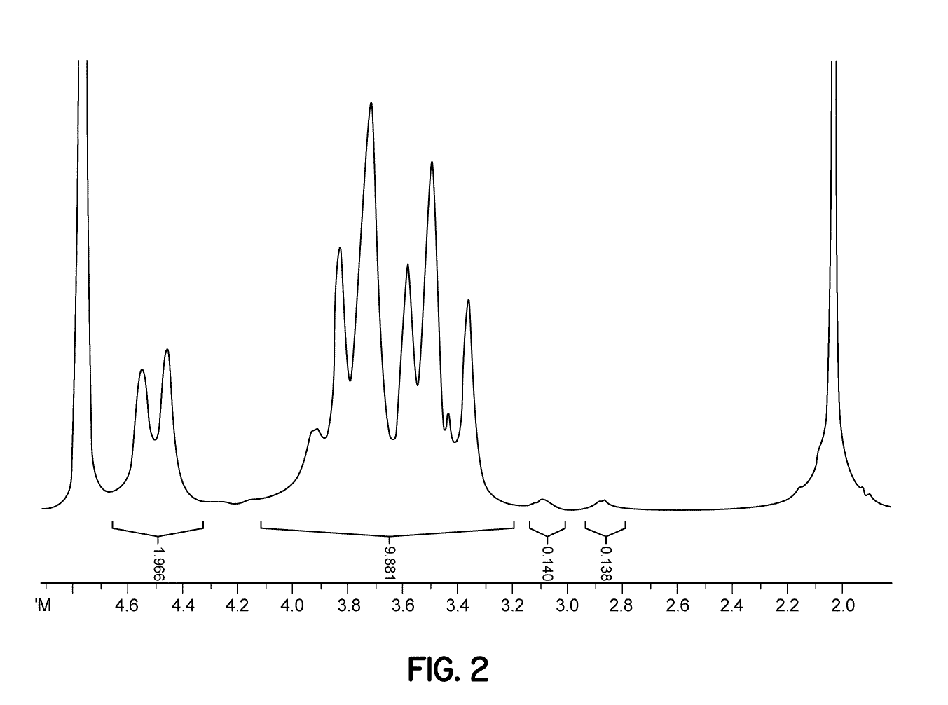

Thirdly, a cycloaddition reaction is carried out; in order to perform it, the polymer must have at least one azide or alkyne group per macromolecule. The degree of chemical modification using the “oxidation-reductive amination” procedure varies from 8 till 30%, depending on the reaction conditions. FIG. 1 shows the ¹H NMR spectra of chemically modified HA bearing a propargyl moiety and DS of 14%. FIG. 2 shows the ¹H NMR spectra of chemically modified HA bearing a 3-azido propyl 2-amine moiety and DS of 15%. The preferred degree of substitution is 10-15%, which is obtained during the reaction in one step procedure. In further embodiments the degree of substitution may be selected in the range from 2 to 30, such as 5, 10, 15, 20, 25 and 30.

The cycloaddition reactive moieties are connected to the polymeric backbone by a linker comprising a stable secondary amine bond; the obtention thereof was described above. As used herein, a secondary amine is represented by the formula —C—NH—R. The introduction of such moieties into macromolecules bonded by secondary amines or imines represents a second aspect of this invention.

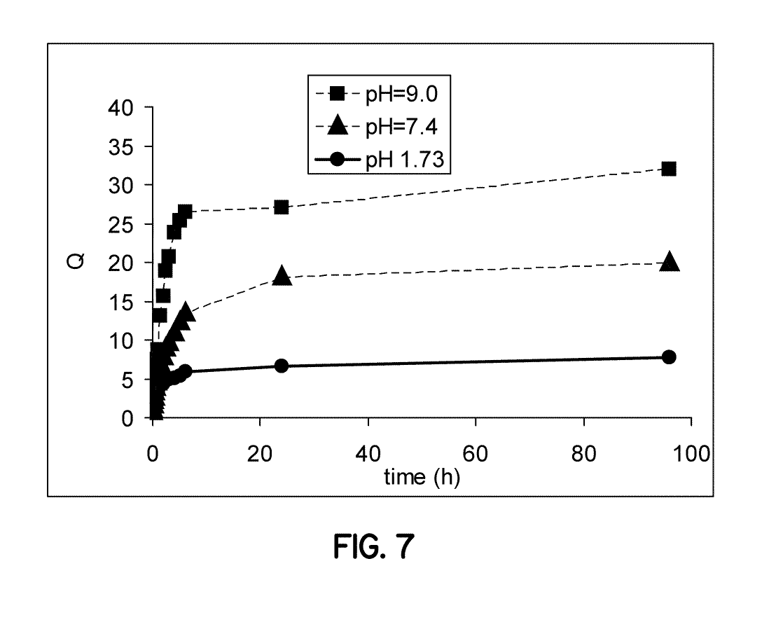

Amphoteric secondary amines incorporated into a polymer network (FIG. 1) had provided the network with stronger interactions within the material, those can include hydrogen bonding and hydrophobic, thus allowing us to influence the stability of the resulting hydrogels (van Bommel et al., 2004). The introduction of pH-sensitive groups into this click chemistry hydrogel, allows tuning of properties at the molecular level and a reversible switching from shrink to swollen as a response to changes of pH (FIG. 7). This property allows the production of controlled release formulations which can be used for oral administration according to (Qiu & Park, 2001), or as biosensors or permeation switches (Hoffman, 1995).

The material prepared in this fashion shows a highly porosity and good wall interconnectivity. According to scanning electronic microscopy (SEM) the hydrogel structure is a honeycomb-like. The properties of the material can be modified varying experimental parameters such as molecular weight of hyaluronan, degree of substitution and gelation time. Different structures are obtained varying the above described parameters as shown in FIGS. 18, 19 and 21.

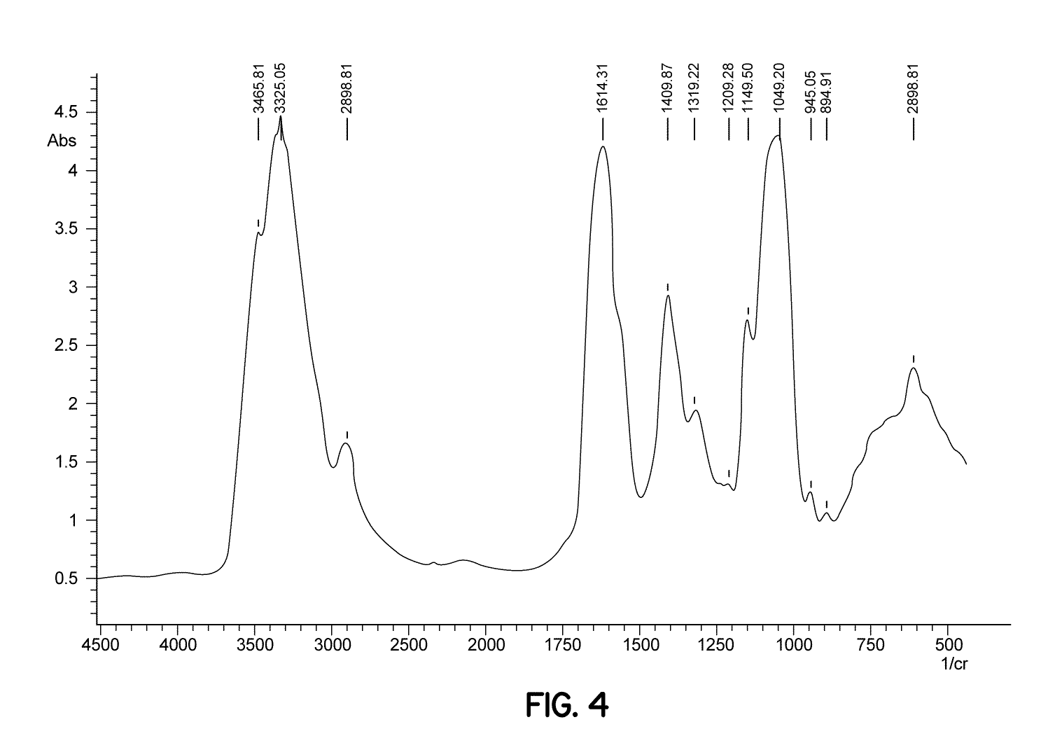

Using the amphoteric material reported in this patent application, it is possible to trigger the release of incorporated agents, sizing the porosity and stability of the resulting material. Intermolecular hydrogen bonding between the secondary amine moieties contributes to a higher stability of the hydrogel. As evidenced from the FT-IR spectra of the freeze-dried materials (xerogels), the NH signals are characteristic of hydrogen bonded secondary amines. (3320-3270 cm-1), whereas the signals originating from the COO⁻ and NH₂⁺ moieties all fell between 1670-1610 cm-1 (FIG. 4).

The material described in this patent application is able of an intake almost two fold the maximum amount of water in comparison to the material reported by Crescenzi et al. (WO/2008/031525-A1). Water uptake proved the existence of strong dipole-dipole interactions into the network. Furthermore, the hydrogels based on materials based on II and III favor cells cultivation due to their high porosity and organized walls. As is known, the void space within the scaffold network is necessary to allow tissue growth, and in order to allow a fast diffusion of nutrients and waste products.

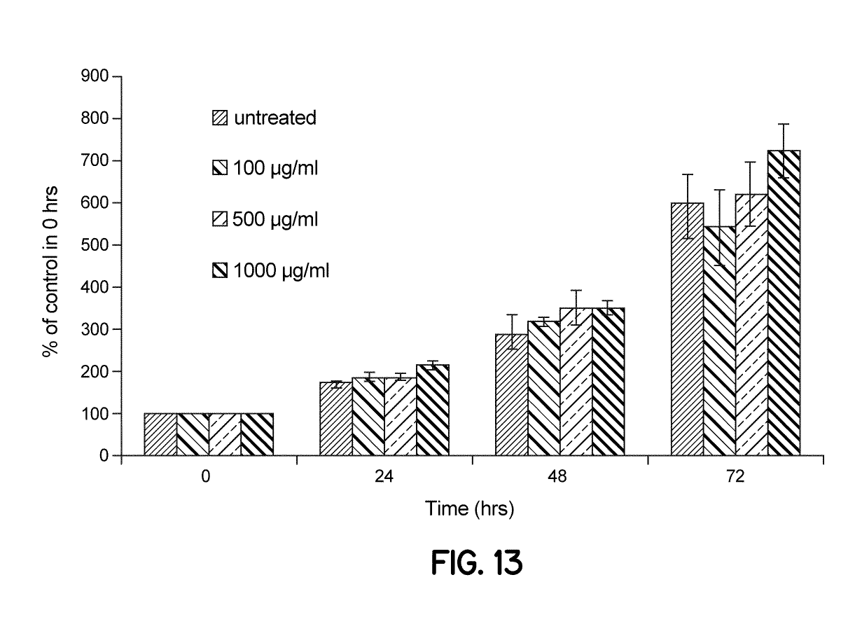

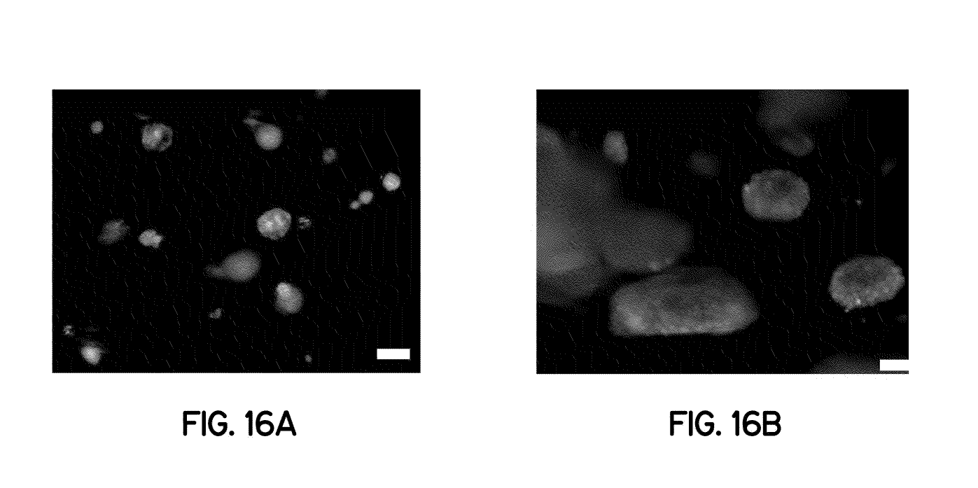

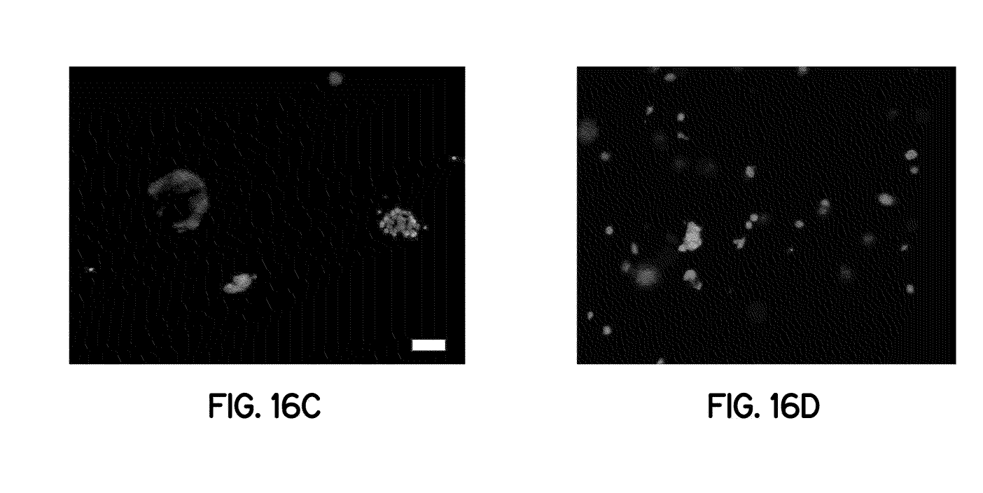

Materials were tested as non cytotoxic and biodegradable (FIGS. 13, 14 and 15). The hydrogels were incubated with chondrocytes and were found as an effective matrix for scaffolds or further applications in tissue engineering (FIGS. 16a and b). The fact that the hydrogels are made of hyaluronic acid which is a biodegradable biopolymer, ensures its biocompatibility, demonstrated by biological testing.

In addition to the total porosity, important factors to consider are the pore size and the interconnectivity. The cross-sectional interior of the swollen hydrogels exhibited flat and interconnected pores (FIGS. 18-21). The pores may present irregular shape, dependent on reaction conditions. However, in all cases walls are interconnected.

Hyaluronan based materials possessing void space has the ability to incorporate drugs and growth factors. It is important to point out that the hydrogels do not present a continuous porous structure by virtue of the freeze-drying step process, within the formation of pores being a result of ice crystal formation and expulsion of the water molecules entrapped in the bulk of the hydrogels.

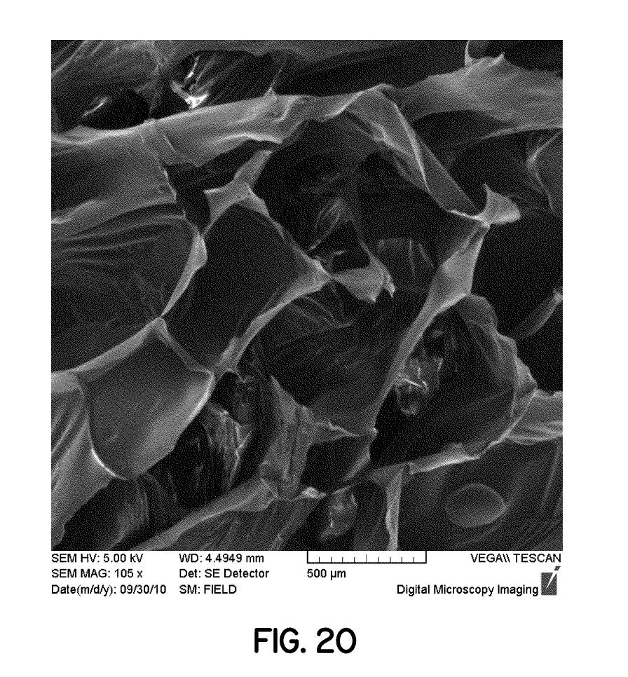

On the other hand this kind of materials resembled natural networks (honeycomb structure). Pore diameters had been determined as 200-300 μm in average. Pilot studies showed that autoclaving did not alter scaffold geometry or induce degradation. The introduction of covalent crosslinking has produced three-dimensional networks. Furthermore, the changes in the hydrogel morphology were evaluated after 30 days of in vitro degradation by surface electron microscopy. Specimens were freeze-dried in liquid nitrogen in order to maintain the original structure. All gels displayed good stability over time, as no changes were observed after two months in different pH buffers. Those results were concluded based on SEM. The structure had been preserved due to the stability of the secondary amines introduced into the material (FIG. 20).

There are three key criteria being considered in the design of this new hydrogel, which have been proved to be fulfilled:
i) Secondary amine bonds are stable to degradation;
ii) The materials described in this patent have not presented any chemical modification in the carboxylic group of hyaluronic acid which is known to be the recognition site of hyaluronidase and HA receptors. Therefore these materials are biocompatible, as demonstrated with the chondrocytes cultivation described in example 15;
iii) The constituents of the crosslinked hyaluronan derivative were tested as non-toxic so they present good biocompatibility and controllable hydrogel composition.

Moreover, we believe that the amphoterism of the material described in this patent is the clue for the prolonged and complete release of substances (controlled sustained release). This is explained by possible supramolecular interactions that control the release from the hydrogel which acts as a microcarrier. Interactions and geometry of the hydrogel govern the diffusion of the molecule through the hydrogel (Kim & Peppas, 2002). Although several researchers have studied the effect drug polymer interactions on molecular release, no mathematical model has been described for predicting the behavior. Moreover, the amphoterism of the modified HA causes a self-assembly and intermolecular bonds which produced a higher and organized porosity in the hydrogel. The organized morphology described in this kind of hydrogels is unique and independent of the concentration but more dependent of self assembling, which is explained by electrostatic interactions (dipole-dipole). During the course of development it was discovered that the entrapment of drugs in the network is neither controlled by the degree of crosslinking, nor by the drug concentration (as described by Crescenzi et al) but by the acido-base Lewis interactions between the amphoteric secondary amines-containing hydrogels and the dipole moment presented on the molecular structure of different drugs. It is very well documented in literature that amides are very weak bases, while the conjugate acid of an amine has a pKa of about 9.5; the conjugate acid of an amide has a pKa around −0.5. As a result, amides present poor acido-base properties in water. This lack of basicity is explained by the electron-withdrawing nature of the carbonyl group where the lone pair of electrons located is delocalized by resonance. The material described in the present patent contains secondary amines; the presence of N—H dipoles allows an amphoteric function forming hydrogen bond donors as well as acceptors.

Our methodology offers a clear advantage compared to the amides previously reported in the patent application WO/2008/031525-A1. Table 1 shows the comparative data of the properties of the materials reported in WO2008/031525 “Hyaluronic acid derivatives obtained via click chemistry crosslinking” and of the amphoteric materials based on hyaluronic acid, according to the invention, possessing a porous morphology and tailored microstructure. More specifically, secondary amines can participate in hydrogen bonding with water more actively than amide derivates. As a result of such acido-base interactions, the produced material is able to absorb higher amounts of water, therefore act as a preferable moisturizing agent. Furthermore, the interconnected pores have allowed fast absorption of water by diffusion. This patent application discloses a method to prepare hydrogels which are able to overcome slow absorption of water into the glassy hydrogel because they present higher diffusion ability. More specifically, Table 1 resumes and compares the release of benzydamine hydrochloride from the material crosslinked via click chemistry and reported in the patent application WO2008/031525 and the amphoteric materials based on hyaluronan as described in this patent application.

TABLE 1

| | Media | | | | | |
|---|---|---|---|---|---|---|
| | t₅₀ (h) | | t₇₀ (h) | | t ₈₈ (h) | |
| Derivative | WO | AM | WO | AM | WO | AM |
| water | 1 | 8 | 5 | 15 | ND | 24 |
| PBS pH = 7.4 | 1 | 2 | 2 | 4 | 3.5 | 8 |

AM=Amphoteric hydrogel based on compounds (II) and (III) described in this patent application, wherein R₁ is methyl and R₂ is propyl; WO=material according to WO2008/031525; ND=not determined; t₅₀ represents the time (in hours) in which 50% of benzydamine hydrochloride is released, t₇₀ represents the time (in hours) in which 70% of benzydamine hydrochloride is released, and t₈₈ represents the time (in hours) in which 88% of initial incorporated benzydamine hydrochloride is released.

The material reported in WO2008/031525 retains only for one hour 50% of the initial benzydamine hydrochloride incorporated in the gel before gelation, independent of the release media. On the other hand, 50% of the same drug was retained in the amphoteric material for 8 hours using water as a release media, which indicates a smart hydrogel. Moreover 70% of benzydamine is released from the WO material after 5 hours in water, whereas the same amount is released from the amphoteric material of the invention in this action after 15 hours, which means that the hydrogel is able to create stronger electrostatic interactions between charged molecules inside the network. In PBS the AM material had shown the same behavior, higher retention time. Table 1 shows that Crescenzi's material (WO) had released 88% of the original incorporated drug after 3.5 hours, wherein the AM material after 8 hours.

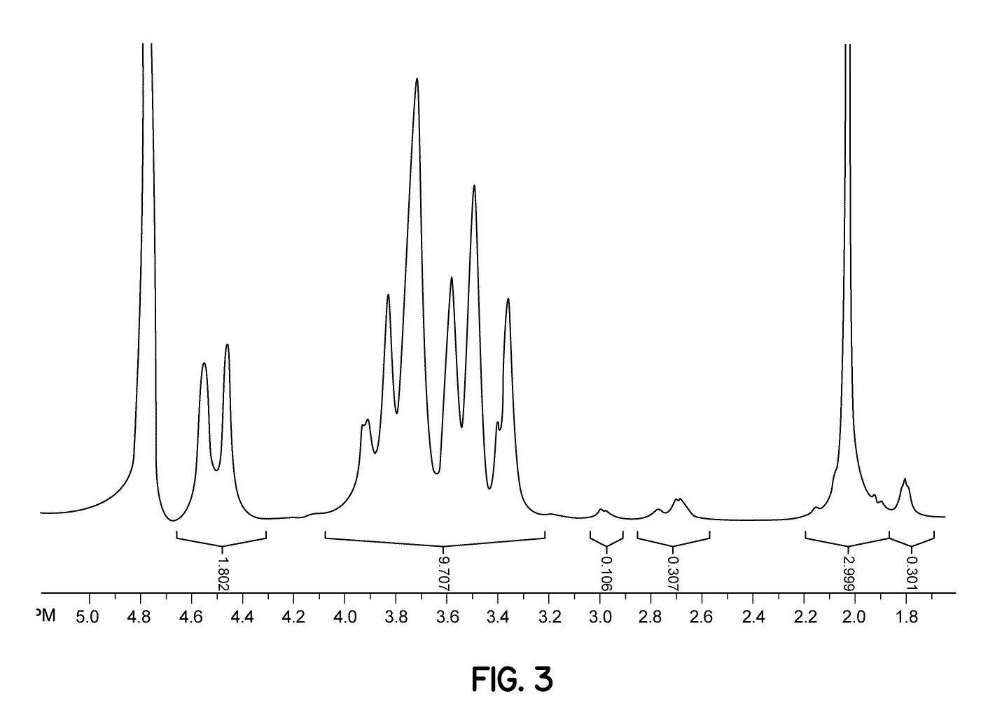

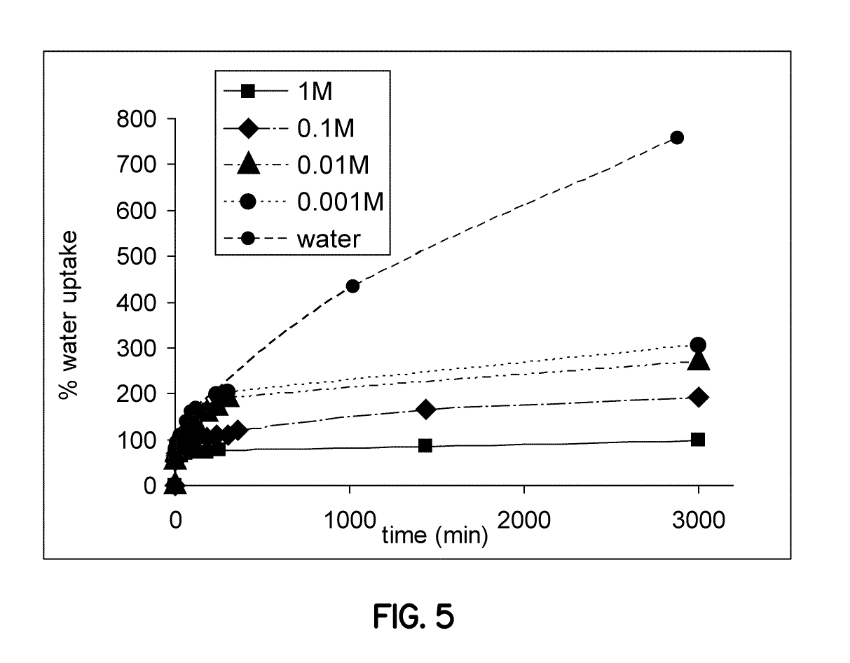

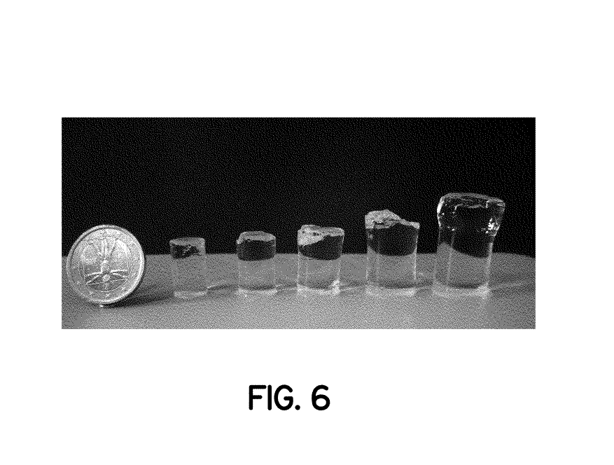

An important characteristic of the hydrogels according to the present invention consists in the fact that the polymer network has both pendent —COOH groups (compositional non modified hyaluronic acid dimers) and backbone secondary amines (—N—) that can impart amphoteric pH-sensitivity to the hydrogel (FIG. 1). Herein, we reported the synthesis, characterization (FIGS. 2, 3 and 4) and swelling behavior (FIGS. 5, 6 and 7) of amphoteric hydrogels based on II and III where R₁ is methyl and R₂ is propyl. FIG. 5 shows the plot describing the water uptake of hydrogels based on II and III where R₁ is methyl and R₂ is propyl (For samples shown physically in FIG. 6). The gels (without initial drying) were allowed to swell for 48 hours at room temperature in media of different and controlled ionic strength and the results are resumed in FIG. 6. FIG. 5 shows the maximum water volume absorbed by the material is 700% of their initial weight. For all the examples, the hydrogels had reached a maximum degree of swelling and after that point the gel started bio-degradation.

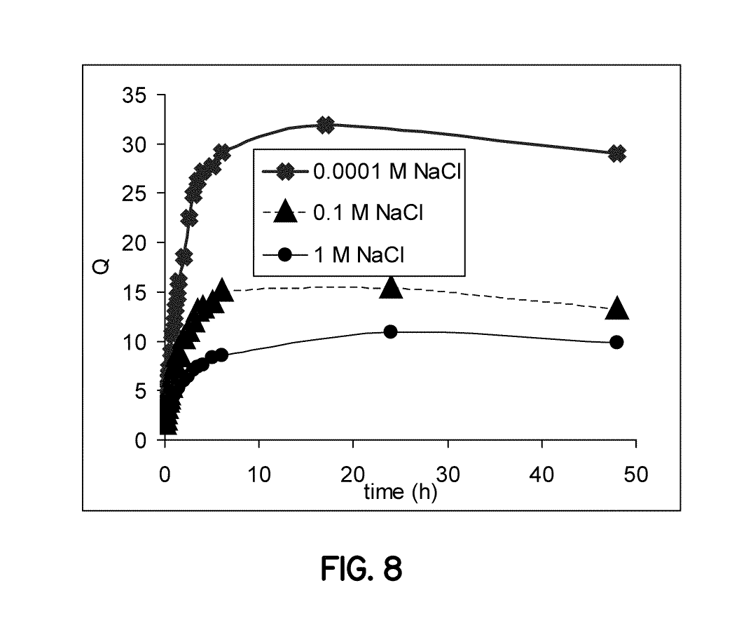

For a pH-sensitivity test, the dried hydrogels were placed in media with various pH values at 37° C. for 48 hours to reach the swelling equilibrium. The hydrogels were took out, wiped with a moistened filter paper and weighed every two hours till the equilibrium was reached. Swelling kinetics studies were carried out as well. The dried hydrogel samples (xerogels) were immersed in media characterized by different pH=1.73 (below pKₐ of HA), 7.4 and 9.0 at 37° C. for a predetermined time, then wiped with a moistened filter paper and weighed. The swelling media of varying pH values were prepared using as reference european pharmacopeia, the ionic strength was kept at I=0.1 M using NaCl in both for the swelling kinetics and the pH sensitivity tests (FIGS. 7 and 8). All the above experiments were carried out in triplicate, and the swelling ratios are reported as the average of three separate experiments±SD (n=3).

The swelling capacity of the hydrogels (Q), defined as the ratio between the weight of the swollen gels (Ws) after extensive dialysis against distilled water or NaCl solution in different ionic strength and the weight of the dry networks was studied and resumed in FIG. 8. Samples were swollen in distilled water or different ionic strength solution at 25° C. until equilibrium (constant weight) was reached.

For a pH sensitive hydrogel, water transportation into the hydrogel is the first indispensable step before the hydrogel can function as a sensor or a controlled release system. Therefore, the clear knowledge of the water transport mechanism in a hydrogel and the induced hydrogel swelling mechanism, is necessary for directing the selection of appropriate drugs to be loaded and for controlling the drug release and rate. FIG. 7 illustrates the effect of the pH values on the swelling kinetics of amphoteric hydrogels based on hyaluronic acid, expressed as a function of the swelling ratio vs. immersion time of the hydrogel. Swelling kinetics was investigated in different media pH=1.73 (below pKa of HA), phosphates-saline buffer pH=7.4 and pH=9.0 at 37° C. The specific pH values were chosen since in these values the hydrogels had shown the highest swelling degree. A second weight of calculate swelling degree as described in this patent was obtained by volume determination; for this experiment the volume occupied by 1 g of solution of 1% w/v concentration was determined experimentally as 474±20 mm³ using the cylinder equation. After 24 hours of swelling in PBS-saline buffer at room temperature the hydrogel had increased volume to 677±46 mm³. After 96 hours of swelling in the same media, the hydrogels have presented the volume of 633±44 mm³. Using the standard deviation it is possible to believe that the hydrogel had lost volume, maybe due to biodegradability. When the material is applied in-vivo, we could suppose that natural hyaluronidase will increase the degradation process.

The swelling kinetics of hydrogels can be approximately expressed using the equation:

(Ws−Wd)/(We−Wd)=ktⁿ Eq (2)

Where Wd, Ws and We are the weights of dried (d), swollen (s) hydrogels at time t and reaching the equilibrium (e), respectively, k is a constant related to the hydrogel network structure, and n is a swelling exponent indicating swelling mechanism. If n≦0.5, the hydrogel follows a Fickian diffusion, in which water transport is governed by a simple concentration gradient. If n=1, then the water uptake is controlled by convection, where hydrogel relaxation is the predominant mechanism for the water transport. If n is between 0.5 and 1, then the water uptake will conform by anomalous diffusion, where the water diffusion rate is comparable to the polymer relaxation rate. To gain insight into the swelling mechanism, curves of swelling degree against time were plotted in logarithmic scale (data not shown) and a linear fitting was conducted in the initial phases of Ws/We≦60% according to Peppas theory of water transport into a hydrogel (Ritger & Peppas, 1987a, b). The obtained n values and the coefficients of determination (R²) are summarized in Table 2.

TABLE 2

| pH value | n | R² |
|---|---|---|
| 1.7 | 0.47 | 0.991 |
| 7.4 | 0.60 | 0.997 |
| 9.0 | 0.73 | 0.994 |

Table 2 resumes the n values at different pH. Phosphate-saline buffer of pH=7.4 was the first to be evaluated. The calculated n=0.6 and 0.73 values in physiological pH and pH=9.0 respectively, suggests that the swelling follows an anomalous diffusion. The calculated value of n=0.47 on pH=1.7 means that the hydrogel follows a Fickian diffusion. As indicated in FIG. 1 both —NH₂—⁺ cations and —COO⁻ anions are present in the hydrogel at pH=7.4, the presence of dipoles favors an additional formation of hydrogel bonds into the hydrogel, which consequently prevents water diffusion and becomes the rate-limiting step for swelling. At lower pH=1.7 where are —NH₂⁺— cations and —COOH are protonated, the hydrogel shrinks. Strong electrostatic repulsions inside the hydrogel produced a swollen hydrogel (See FIG. 7, pH=9.0). The repulsions enhance the rigidity and slow down the relaxation. In order to obtain a zero order drug release, the properties of the drug to be-loaded and hydrogel size and shape should also be considered.

The biomaterial according to the invention may be in the form of a scaffold for cell cultures, as well as a gel containing cellular material for use in tissue engineering or regeneration.

Further embodiments will become obvious from the following examples which illustrate the invention in some of its mayor aspects, without limiting the scope thereof.

### EXAMPLES

### Example 1

### Preparation and Isolation of Oxidized-Hyaluronan (HA-Ox)

10 g of hyaluronan with different molecular weight corresponding to 125 molar equivalents of a monomeric unit are solubilized in 5000 ml of water at room temperature for 24 hours prior to the experiment. 19.2 g of KBr (16.5 mmol) and 10 g of NaH₂PO₄ were added to this solution. The reaction mixture was cooled to 0° C. The reaction was evacuated and refilled with nitrogen. Then a water solution containing 10.7 mg of 4-acetamido-TEMPO (0.05 mmol) was added to the reaction mixture, followed by the addition of 0.588 mL (1.25 mmol) of sodium hypochlorite, corresponding to 10% of the moles of sodium hyaluronate present. The oxidation reaction is carried out for 2 hours at 5° C. The solution is then diluted with 5000 ml of water and the pH adjusted to 7.0. The solution was ultrafiltrated using a centramate cassette (Paal Co) with a molecular cut-off of 10 kDa. The product was precipitated with IPA and washed three times with IPA:water (100:0, 80:20, 60:40). The precipitate is dried in the oven at 60° C.

The reaction product thus obtained was fully characterized by analytical methodologies. Yield of the reaction: 90-99%. The molecular weights of the obtained products are described below in Table 3 and Table 4 as Mw (_{oxidized HA}) as they were used for a posterior chemical modification.

FT-IR (KBr, cm-1): 3419 (υ, —O—H), 2923, 2160, 1652, 1614, 1413, 1080, 1039, 611.

NMR ¹H (500 MHz, NaOD, δ ppm): 3.4-4.0 (m, 10H), 4.5 (d, 2H), 5.2 (s, 1H)

DOSY:
log D (2.03 ppm CH₃—CO—NH-Polymer)˜−10.5 m²/s
log D (5.2 ppm —CH₂NH—R)˜−10.5 m²/s

### Example 2

### Preparation of HA-Propyl Azidoamine (II)

First, the linker 3-azidopropanamine was synthesized in the following way. 3-chloro-propylamine hydrochloride (1.00 g) and sodium azide (2,5016 g, 3 eq) were dissolved in water (10 mL), followed by an addition of a catalytic amount of KI. The flask was attached to a water condenser and the reaction mixture was heated at 90° C. for 72 hours. After cooling to room temperature, sodium hydroxide was added until pH 11. The free amine was extracted from the reaction mixture employing ether. The organic fraction was dried with sodium sulfate and concentrated under vacuum avoiding complete dryness. Amino-azides of short carbon chain are suspected explosives. The ¹H NMR confirmed the structure and purity of the compound. NMR (500.13 MHz, CDCl₃); 1.41 (2H, bs, —NH₂), 1.76 (2H, q, J=6.8), 2.81 (2H, t, J=6.8), 3.38 (2H, t, J=6.8). FT-IR (KBr, cm-1): 3363, 2941 (υ —CH₂), 2100 (υ —N≡N), 1650, 1593, 1461, 1286.

Next, HA-propyl azidoamine (HA-2APA) with a degree of substitution of 15% was prepared by coupling the linker (3-azidopropylamine) to oxidized hyaluronic acid. Detailed conditions for preparation are given for a number of examples in Table 3. In general, oxidized hyaluronan was dissolved in a round bottom flask in 50 ml of acetate buffer (pH=6.0) at room temperature. After 24 hours of stirring, 3-azidopropylamine was added and the reaction proceeded at room temperature for the time given in Table 3 as time for imine formation. Then picoline-borane was added to the reaction mixture and the reaction was carried out for the time given in Table 3 as time for reduction, diluted with 50 ml of water, dialyzed against 5% solution of NaHCO₃/NaCl, and either freeze-dried or ultrafiltered and dried in the oven at 60° C. The yields of the reaction vary from 80-95%. Polydispersity and the respective molecular weight of the derivatives are resumed in Table 3.

FT-IR (KBr, cm-1): 3379 (υ, —O—H), 2894, 2008 (υ, N₃), 1614, 1407, 1078, 613.

NMR ¹H (500 MHz, NaOD, δ ppm): 2.0 (s, 3H), 2.85 (m, 2H), 3.1 (m, 2H), 3.4-4.0 (m, 10H), 4.5 (d, 2H).

DOSY

log D (2.03 ppm CH₃—CO—NH-Polymer)˜−10.5 m²/s

log D (2.7 ppm —CH₂NH—R)˜−10.5 m²/s

log D (3.0 ppm —CH₂NH—R)˜−10.5) m²/s

TABLE 3
Reaction conditions used for the synthesis of HA-azido propyl-2-amine (HA-
CAPA)

| | Mw_{Oxidized HA} | m_{Oxidized HA} | nₗᵢₙₖₑᵣ (eq | n_{reductive agent} | | T_{reduction} | | Mw_{HA-CAPA} |
|---|---|---|---|---|---|---|---|---|
| entry | (kDa)/P | (g) | HA) | (eq HA) | Tᵢₘᵢₙₑ (h) | (h) | DS (%) | (kDa)/P |
| 1 | 108/1.3 | 0.2 | 0.5 | 3 BCN | 24 | 24 | 15 | 30/1.4 |
| 2 | 300/1.5 | 0.4 | 1 | 3 BCN | 72 | 48 | 15 | 300/1.8 |
| 3 | 300/1.5 | 0.4 | 1 | 3 PB | 72 | 48 | 15 | 31/1.4 |
| 4 | 316/2.0 | 2.0, | 1 | 1 PB | 72 | 48 | 15 | 90/1.4 |
| 5 | 612/1.9 | 2.0, | 1 | 1 PB | 72 | 48 | 15 | 89/1.4 |
| 6 | 612/1.9 | 10.0 | 0.4 | 0.2 PB | 6 | 8 | 15 | 124/1.4 |
| 7 | 755/1.46 | 0.8 | 0.5 | 3 BCN | 24 | 72 | 15 | 170/1.6 |

Mw = molecular weight
m = weight
n = molar amount
Tᵢₘᵢₙₑ = time of imine formation
T_{reduction} = time for imine reduction
PB = picoline borane
BCN = sodium cyanoborohydride
eqHA = equivalents in respect to HA dimer
DS = degree of substitution
P = polydispersity

### Example 3

### Preparation of HA-Propargyl Amine (HA-CAPr)

Detailed conditions for preparation are given for several of examples are resumed in Table 4. In general, oxidized hyaluronan prepared in example 1 was dissolved in 40 ml of water at room temperature for 24 hours. An amount of propargylamine given in Table 4 was added into the reaction mixture to form imine. The formation of imine was allowed for a time specified in Table 4 (T imine) while stirring the reaction solution at room temperature. After this time, a 1% aqueous solution of reductive agent (Table 4) was added to the reaction mixture and the reaction was allowed to proceed for a period assigned as T reduction (Table 4). The reaction mixture was then diluted with 50 ml of water, thoroughly dialyzed against water and either freeze-dried or ultrafiltered and dried in oven at 60° C. (Table 4).

FT-IR (KBr, cm-1): 3379 (υ, —O—H), 2894, 2131 (υ, C≡C), 1614, 1407, 1078, 613.

NMR ¹H (500 MHz, NaOD, δ ppm): 2.0 (s, 3H), 2.85 (m, 2H), 3.1 (m, 2H), 3.4-4.0 (m, 10H), 4.5 (d, 2H).

DOSY

log D (2.03 ppm CH₃—CO—NH-Polymer)˜−10.5 m²/s

log D (2.7 ppm —CH₂NH—R)˜−10.5 m²/s

log D (3.0 ppm —CH₂NH—R)˜−10.5) m²/s

TABLE 4
Reaction conditions used for the synthesis of HA-CAPr. (Scheme 3)

| | MW_{Oxidized HA} | m_{Oxidized HA} | n_{propargylamine} | n_{reduction} | | T_{reduction} | | MW_{HA-CAPr} |
|---|---|---|---|---|---|---|---|---|
| entry | (kDa)/P | (g) | (eq HA) | (eq HA) | Tᵢₘᵢₙₑ (h) | (h) | DS (%) | (kDa)/P |
| 1 | 108/1.3 | 0.2 | 0.5 | 3 BCN | 24 | 24 | 19 | 94/1.6 |
| 2 | 108/1.3 | 0.2 | 0.5 | 3 BCN | 24 | 24 | 19 | 31/1.4 |
| 3 | 300/1.5 | 0.4 | 1 | 3 BCN | 72 | 48 | 21 | 229/1.4 |
| 4 | 300/1.5 | 0.4 | 1 | 3 PB | 72 | 48 | 21 | 165/1.7 |
| 5 | 612/1.9 | 2.0 | 1 | 1 PB | 72 | 48 | 15 | 199/1.6 |
| 6 | 612/1.9 | 2.0 | 1 | 1 PB | 72 | 48 | 15 | 199/1.6 |
| 7 | 612/1.9 | 10 | 0.4 | 0.4 PB | 6 | 48 | 12 | 361/1.5 |
| 8 | 612/1.9 | 10 | 0.2 | 0.2 PB | 6 | overnight | 15 | 124/1.4 |

### Example 4

### Oxidation of Hyaluronic Acid Free of Sodium, Oxidation and Reductive Amination

In brief, 5 g of Hyaluronic acid of a mean molecular weight of 1.2 MDa was dissolved in 500 ml of distilled water. A DOWEX 50WX8 resin cation resin exchange (H type) was added to the mixture. After ion exchange, the resin was removed by centrifugation at 5000 rpm for 5 minutes and the resulting solution was frozen at −80° C. and lyophilized. The molecular weight and the polydispersity of the polymer after the cationic exchange were determined by SEC-MALLS.

1.0 g of Hyaluronic acid (free from sodium) of a mean molecular weight of 100 kDa is dissolved for 24 h at 60° C. in 100 ml of DMSO. 0.35 g of Dess-Martin Periodinane corresponding to 0.8 eq of hyaluronic acid was added to the reaction mixture. The reaction is carried out for 6 h at room temperature. The reaction mixture was isolated by precipitation. In brief, the reaction mixture is slowly poured into 2-propanol (500 ml). A precipitate is formed which is filtered and washed four times with 200 ml of 2-propanol/DMSO 80:20 and three times with 2-propanol (100 ml). The product was vacuum dried overnight at 60° C. The product is dissolved in water and slowly poured into 2-propanol under constant stirring. The precipitate is isolated and characterized. The degree of substitution by this methodology was calculated as DS=28% (DS=[CH₃CONH/NH—CH/].)

FT-IR (KBr, cm-1): 3419 (υ, —O—H), 2923, 2160, 1710, 1652, 1614, 1413, 1080, 1039, 611.

NMR ¹H (500 MHz, NaOD, δ ppm): 3.4-4.0 (m, 10H), 4.5 (d, 2H), 5.2 (s, 1H)

DOSY:

log D (2.03 ppm CH₃—CO—NH-Polymer)˜−10.5 m²/s

log D (5.2 ppm —CH₂NH—R)˜−10.5 m²/s

### Obtention of Reduced Derivatives

The general synthetic procedure was carried out as described next. 1 g of oxidized hyaluronic acid was dissolved in water overnight (100 ml). To this solution propargyl amine was added, the respective amine corresponding to 0.46 eq to hyaluronic acid present in the reaction. The solution was stirred 48 h at room temperature. 1 eq. of a solution of NaBH₃CN in water 1% w/w was prepared and added to the reaction mixture. The mixture was stirred for 96 h at room temperature. After completion of the reaction, the solution was dialyzed against mixture of 0.1% NaCl and 0.1% NaHCO₃ and extensively against water. The final product was isolated by lyophilization.

Reduced Product: Propargyl Derivative

DS=28% (DS=[CH₃CONH/NH—CH/].

FT-IR (KBr, cm-1): 3379 (υ, —O—H), 2894, 2131 (υ, C≡C), 1614, 1407, 1078, 613.

¹H NMR (500 MHz, NaOD, δ ppm): 2.0 (s, 3H), 2.85 (m, 2H), 3.1 (m, 2H), 3.4-4.0 (m, 10H), 4.5 (d, 2H).

DOSY

log D (2.03 ppm CH₃—CO—NH-Polymer)˜−10.5 m²/s

log D (2.7 ppm —CH₂NH—R)˜−10.5 m²/s

log D (3.0 ppm —CH₂NH—R)˜−10.5) m²/s

Molecular weight of the isolated product determined by SEC MALS=15 kDa and polydispersity=1.5

Reduced Product: Azido Derivative

DS=28% (DS=[CH₃CONH/NH—CH/].

FT-IR (KBr, cm-1): 3415 (υ, —O—H), 2921, 2111 (υ, N₃), 1614, 1407, 1377, 1078, 613.

¹H, HSQC, NMR (500 MHz, NaOD, δ ppm): 2.0 (s, 3H), 2.014 (s, 3H) 2.85 (m, 2H), 3.08 (t, 2H J=10.95), 3.4-4.0 (m, 10H), 4.45 (dd, 1H), 4.53 (dd 1H).

DOSY

log D (2.03 ppm CH₃—CO—NH-Polymer)˜−10.5 m²/s

log D (2.7 ppm —CH₂NH—R)˜−10.5 m²/s

log D (3.0 ppm —CH₂NH—R)˜−10.5) m²/s

Molecular weight of the isolated product determined by SEC MALS=15 kDa and polydispersity=1.5

### Example 5

### Oxidation of Hyaluronan and Reductive Amination in One Pot for Obtaining the Propargyl Derivate

Detailed conditions for preparation are given for a number of examples in Table 5. In general, 10.0 g of sodium hyaluronate with different molecular weights as described in Table 5 as MW_{HA} was dissolved in 960 ml of water. To that solution was added 2.57 g of sodium bromide. (2.5 mmol. 38.8 g of sodium phosphates was added to the reaction mixture in order to reach pH=9.0. The following reactants were added in sequence: 53.3 mg of 4-acetamido-TEMPO previously dissolved in water (1 ml) and subsequently 3.0 ml of sodium hypochlorite. The mixture was left under stirring for the time described as T imine (Table 5). The reaction was allowed to reach room temperature. In this moment, the pH was adjusted potentiometrically with addition of acetic acid to reach pH=5.5. To this mixture propargyl amine was subsequently added, 0.3 eq. corresponding to hyaluronic acid present in the reaction mixture. The reductive amination was carried out for the time described in Table 5. In this moment, 0.424 g of picoline borane was added, corresponding to 0.3 eq. of hyaluronic acid. The reaction was allowed to proceed at room temperature overnight. The product was purified by ultrafiltration. The propargyl-modified HA product was fully characterized by common analytical techniques. The signals used for the quantitative evaluation of propargyl amine moieties bounded to HA are the methyl assigned to HA in comparison to the methylene assigned to the modified polysaccharide.

DS=[described in Table 5 CH₃CONH/NH—CH/].

FT-IR (KBr, cm-1): 3379 (υ, —O—H), 2894, 2131 (υ, C≡C), 1614, 1407, 1078, 613.

NMR ¹H (500 MHz, NaOD, δ ppm): 2.0 (s, 3H), 2.85 (m, 2H), 3.1 (m, 2H), 3.4-4.0 (m, 10H), 4.5 (d, 2H).

DOSY

log D (2.03 ppm CH₃—CO—NH-Polymer)˜−10.5 m²/s

log D (2.7 ppm —CH₂NH—R)˜−10.5 m²/s

log D (3.0 ppm —CH₂NH—R)˜−10.5 m²/s

TABLE 5
Reaction conditions used for the synthesis of HA-CAPr (Scheme 3)

| | | m_{Oxidized HA} | | n_{reductive agent} | | | | |
|---|---|---|---|---|---|---|---|---|
| | MW_{HA} | (g, | nₗᵢₙₖₑᵣ (eq | (eq | Tᵢₘᵢₙₑ | T_{reduction} | | MW_{HA-CAPr} |
| Entry | (kDa)/P | % w/w) | HA) | HA) | (h) | (h) | DS (%) | (kDa)/P |
| 1 | 19 | (2.0, 1) | 0.3 | 0.3 | 5 | overnight | 12 | 17/1.3 |
| 2 | 19 | (2.0, 1) | 0.3 | 0.3 | 5 | overnight | 12 | 17/1.3 |
| 3 | 90 | (10.0, 1) | 0.3 | 0.3 | 5 | overnight | 8 | 84/1.5 |
| 4 | 90 | (10.0, 1) | 0.3 | 0.3 | 5 | overnight | 8 | 83/1.4 |
| 5 | 90 | (20, 1) | 0.3 | 0.3 | 5 | overnight | 12 | 88/1.5 |
| 6 | 90 | (20, 1) | 0.3 | 0.3 | 5 | overnight | 12 | 84/1.5 |
| 7 | 90 | (20, 2) | 0.3 | 0.3 | 5 | overnight | 12 | 87/1.2 |
| 8 | 90 | (20, 3) | 0.3 | 0.3 | 5 | overnight | 12 | 86/1.2 |
| 9 | 90 | (20, 4) | 0.3 | 0.3 | 5 | overnight | 12 | 86/1.2 |
| 10 | 90 | (20, 5) | 0.3 | 0.3 | 5 | overnight | 12 | 86/1.2 |
| 11 | 202 | (10.0, 1) | 0.3 | 0.3 | 5 | overnight | 15 | 140/1.2 |
| 12 | 498 | (10.0, 1) | 0.3 | 0.3 | 5 | overnight | 15 | 226/1.8 |

PA: Propargyl amine

### Example 6

### Oxidation of Hyaluronan and Reductive Amination in One Pot for the Propargyl Derivate (HA-CAPr) at 4° C. (Lower DS).

Detailed conditions for preparation are given for a number of examples in Table 5. In general, 10.0 g of sodium hyaluronate with a mean molecular weight of 1.8 MDa was dissolved in 960 ml of water. To that solution 2.57 g of sodium chloride (2.5 mmol) was added. 38.8 g of sodium phosphates was added to the reaction mixture in order to reach pH=9.0. The following reactants were added in sequence: 53.3 mg of 4-acetamido-TEMPO previously dissolved in water (1 ml) and subsequently 3.0 ml of sodium hypochlorite was added to the solution. The mixture was left under stirring for 15 minutes at 4° C. The reaction was allowed to reach room temperature. In this moment, the pH was adjusted potentiometrically with addition of acetic acid to reach pH=5.5. To this mixture was subsequently added propargyl amine, 0.3 eq. corresponding to hyaluronic acid presented in the reaction mixture. The reductive amination was carried out for 5 hours. In this moment, 0.424 g of picoline borane was added, corresponding to 0.3 eq. of hyaluronic acid. The reaction was allowed to proceed at 4° C. overnight. The product was purified by ultrafiltration. The propargyl modified —HA product was fully characterized by common analytical techniques. The signals used for the quantitative evaluation of propargyl amine moieties bounded to HA are the methyl assigned to HA in comparison to the methylene assigned to the modified polysaccharide. The molecular weight determined by SEC-MALLS reveals a mean molecular weight of 900 kDa and polydispersity of 1.7.

DS=[circa 5% CH₃CONH/NH—CH/].

FT-IR (KBr, cm-1): 3379 (υ, —O—H), 2894, 2131 (υ, C═C), 1614, 1407, 1078, 613.

NMR ¹H (500 MHz, NaOD, δ ppm): 2.0 (s, 3H), 2.85 (m, 2H), 3.1 (m, 2H), 3.4-4.0 (m, 10H), 4.5 (d, 2H).

DOSY

log D (2.03 ppm CH₃—CO—NH-Polymer)˜−10.5 m²/s

log D (2.7 ppm —CH₂NH—R)˜−10.5 m²/s

log D (3.0 ppm —CH₂NH—R)˜−10.5 m²/s

### Example 7

### Oxidation of Hyaluronan and Reductive Amination in One Pot at 4° C. For Obtention of HA-Azide Propyl Derivative

10.0 g of sodium hyaluronate with a mean molecular weight as described in Table 6 (MW HA) were dissolved in 960 ml of water. The solution was cooled down to 4° C. To that solution 2.57 g of sodium bromide (2.5 mmol) was added. 38.8 g of Na₂HPO₄ was added to the reaction mixture. The pH of the reaction was adjusted to 9.0 with a solution of 0.1 M of sodium hydroxide. The following reactants were then added in sequence: 53.3 mg of 4-acetamido-TEMPO previously dissolved in water (1 ml) and subsequently 3.0 ml of sodium hypochlorite was added to the reaction mixture. The reaction was left under stirring for 15 minutes at 4° C. The reaction maintained at 4° C. had been allowed to reach pH 6.0 with addition of acetic acid. To that mixture azide propyl amine, in the molar ratio as described in Table 6 (nₗᵢₙₖₑᵣ) to the sodium hyaluronate dimers present in the reaction mixture was subsequently added. The reaction was carried out for the time identified in Table 6 as T(ᵢₘᵢₙₑ) (hours). In this moment an amount of picoline borane corresponding to the percentage described in Table 6 as (n_{reductive agent}) was added to the reaction mixture. The reaction was purified by ultrafiltration and precipitated again using different mixtures of IPA: H₂O. The product was dried at 60° C. and the recovery was determined as 95%. SEC-MALLS analyses reveal a mean molecular weight of 800 kDa and polydispersity of 1.6.

TABLE 6
Reaction conditions used for the synthesis of HA-CAPA.

| | | | nₗᵢₙₖₑᵣ | n_{reductive agent} | | | | |
|---|---|---|---|---|---|---|---|---|
| | MW HA | m_{HA} | (eq | (eq | Tᵢₘᵢₙₑ | T_{reduction} | | MW_{HA-CAPA} |
| Entry | (kDa)/P | (g, % w/w) | HA) | HA) | (h) | (h) | DS (%) | (kDa)/P |
| 1 | 90/1.5 | (10.0, 1) | 0.3 | 0.3 | 2 | overnight | 10 | 83 |
| | | | | | | | | 1.4 |
| 2 | 90/1.5 | (20.0, 2) | 0.3 | 0.3 | 2 | overnight | 10 | 85 |
| | | | | | | | | 1.4 |
| 3 | 202/1.5 | (10.0, 1) | 1 | 1 | 2 | overnight | 12 | 127 |
| | | | | | | | | 1.3 |
| 4 | 202/1.5 | (10.0, 1) | 0.3 | 0.3 | 5 | overnight | 12 | 170 |
| | | | | | | | | 1.2 |
| 5 | 498/1.5 | (10.0, 1) | 0.3 | 0.3 | 5 | overnight | 15 | 138 |
| | | | | | | | | 1.6 |

APA: N₃—(CH₂)₃—NH₂

### Example 8

### Oxidation of Hyaluronan and Reductive Amination in One Pot at 4° C. For Obtention of 11-azido-3,6,9-trioxaundecan-2-amine in One Pot

10.0 g of sodium hyaluronate possessing several molecular weights (Table 7) was dissolved in 960 ml of water. The solution was cooled down to 4° C. To that solution 2.57 g of sodium bromide (2.5 mmol) was added. 38.8 g of Na₂HPO₄ was added to the reaction mixture. The pH of the reaction was adjusted to 9.0 with a solution of 0.1 M of sodium hydroxide. The following reactants were then added in sequence: 53.3 mg of 4-acetamido-TEMPO previously dissolved in water (1 ml) and subsequently 3.0 ml of sodium hypochlorite was added to the reaction mixture. The reaction was left under stirring for 15 minutes at 4° C. The reaction maintained at 4° C. had been allowed to reach pH 6.0 with addition of acetic acid. To that mixture was subsequently added 11-azido-3,6,9-trioxaundecan-1-amine, 30% in mol to the sodium hyaluronate dimers in the reaction mixture. After 5 hours at the same temperature, 0.424 g of picoline borane was added, corresponding to 30% mol of the sodium hyaluronate dimers. The product was dried at 60° C. and the recovery was determined as 95%. SEC-MALLS analyses reveal the obtention of molecular weights as described in Table 7.

DS=[circa 10% CH₃CONH/NH—CH/].

FT-IR (KBr, cm-1): 3379 (i), —O—H), 2894, 2008 (—N₃), 1614, 1407, 1078, 613.

NMR ¹H (500 MHz, NaOD, δ ppm): 2.01 (s, 3H), 2.81 (m, 2H), 2.99 (m, 1H), 3.36-3.94 (m, 16H), 4.5 (d, 2H).

HSQC (cross-peak): (2.8-50), (3.4-50), (3.7-70), (3.8-70).

DOSY

log D (2.03 ppm CH₃—CO—NH-Polymer)˜−10.5 m²/s

log D (2.7 ppm —CH₂NH—R)˜−10.5 m²/s

log D (3.0 ppm —CH₂NH—R)˜−10.5 m²/s

TABLE 7
Reaction with 1-azido-3,6,9-trioxaundecan-1-amine
in one pot (HA-CATA)

| | | m_{HA} | Hₗᵢₙₖₑᵣ | | | |
|---|---|---|---|---|---|---|
| | MW HA | (g, | (eq | n_{reductive agent} | DS | MW_{HA-CAPA} |
| Entry | (kDa)/P | % w/w) | HA) | (eq HA) | (%) | (kDa)/P |
| 1 | 90/1.5 | (1.0, 1) | 0.3 | 0.3 | 10 | 89/1.4 |
| 2 | 1300/1.5 | (10.0, 2) | 0.3 | 0.3 | 10 | 268/1.2 |
| 3 | 130/1.5 | (20.0, 1) | 0.3 | 0.3 | 10 | 93/1.5 |
| 4 | 200/1.5 | (20.0, 1) | 0.3 | 0.3 | 10 | 170/1.4 |

### Example 9

### Oxidation of Hyaluronan and Reductive Amination in One Pot. Obtention of 3-azidyl aniline Derivative

5.0 g of aldehyde functionalized hyaluronan with a mean molecular weight of 611 kDa and polydispersity of 1.9 was dissolved in 500 ml of distilled water at room temperature for 24 hours. To this solution 0.308 g of azido-aniline hydrochloride, corresponding to 0.1 eq of the sodium hyaluronate present in the reaction mixture was added. The solution was stirred for 5 h at room temperature. Thereafter, picoline borane suspended in water was added to the reaction mixture, corresponding to 10% mol of hyaluronic acid dimer. The reaction was allowed to proceed overnight at room temperature to yield the HA-azidyl aniline derivative. The reaction was thoroughly dialyzed against water. The obtained polymer was analyzed by the typical analytical techniques. This compound was synthesized in order to as well evaluate the reactivity of different amines to the reductive amination in order to evaluate the efficacy of the crosslinking reaction by electronic effects.

DS=[15% CH₃CONH/NH—CH/].

FT-IR (KBr, cm-1): 3415 (υ, —O—H), 2894, 2121 (υ —N≡N), 1662, 1614, 1407, 1377, 1151, 1078, 615.

NMR ¹H (500 MHz, NaOD, δ ppm): 2.0 (s, 3H), 2.85 (m, 2H), 3.1 (m, 2H), 3.4-4.0 (m, 10H), 4.5 (d, 2H).

DOSY

(2.03 ppm CH₃—CO—NH-Polymer)=7.92×10-12) m²/s

(3.1 ppm-4.2 polymer)˜7.92×10-12) m²/s

(6.85 ppm -o-Ar)˜7.92×10-12) m²/s

(6.95 ppm -m-Ar)˜7.92×10-12) m²/s

### Example 10

### Oxidation of Hyaluronan and Reductive Amination in One Pot. Obtention of 3-Alkynyl Aniline Derivative

1.0 g of aldehyde functionalized hyaluronan with a mean molecular weight of 600 kDa was dissolved in 100 ml of distilled water at room temperature for 24 hours. To this solution 0.250 g of ethynyl aniline, corresponding to 10% mol to the sodium hyaluronate present in the reaction mixture was added. The solution was allowed to stir for 5 h at room temperature. Thereafter, picoline borane suspended in water was added to the reaction mixture, corresponding to 10% in mol to sodium hyaluronate. The reaction was allowed to proceed for 8 h at room temperature to yield the HA-ethynyl derivative. The reaction was diluted with water and thoroughly dialyzed against water. The obtained polymer was analyzed by the typical analytical techniques.

DS=[15% CH₃CONH/NH—CH/].

FT-IR (KBr, cm-1): 3415 (υ, —O—H), 2894, 2121 (υ —N≡N), 1662, 1614, 1407, 1377, 1151, 1078, 615.

NMR ¹H (500 MHz, NaOD, δ ppm): 2.0 (s, 3H), 2.85 (m, 2H), 3.1 (m, 2H), 3.4-4.0 (m, 10H), 4.5 (d, 2H).

DOSY

(2.03 ppm CH₃—CO—NH-Polymer)=−7.92×10-12) m²/s

(3.1 ppm-4.2 polymer)˜−7.92×10-12) m²/s

(6.85 ppm -o-Ar)˜−7.92×10-12) m²/s

(6.95 ppm -m-Ar)˜−7.92×10-12) m²/s.

### Example 11

### Formation of Hydrogel from Modified II and III in Water or PBS

Solutions prepared of derivatives (HA-CAPr) and (HA-CAPA) of different molecular weights varying from 17 kDa and 900 kDa and DS with a molar ratio 1:1 were dissolved overnight (total volume 1.0 ml). The exact amount of reagents was determined based on their degree of substitution (DS) so that the final concentration of both reagents was kept 2% (w/v) in case of DS=100%. Then varying molar amounts of catalyst CuSO₄ and sodium ascorbate were added in the mixture in order to study kinetics of gelation (Table 8). Vortexing was used after the addition of each component to ensure a good homogenization. Then, the reaction solution was stirred for few seconds until the formation of a gel. The gelation time was determined by the vial tilting methodology (Domszy et al, 1986). The prepared gel was dialyzed for 48 h against distilled water or saline phosphates buffer containing 0.01% (w/v) EDTA in order to remove the catalyst. The hydrogels were freeze-dried, and the mass of the freeze dried network was determined. For the SEM experiments the gels were frozen using liquid nitrogen for preserving the original structure and dried. The gel samples were sputtered with gold before SEM analysis.

TABLE 8
Influence of cross-linking reaction conditions of different substituted
HA-CAPA (DS = 8-15%) and HA-CAPr (DS = 8-15%). Reaction
was carried out in a total volume of 1 ml of PBS buffer at 25° C.
All gelation experiments were carried out 5 independent times.

| HA-CAPA | HA-CAPr | | c (Cu²⁺)^{a} | c(SA)^{b} | Gel. Time (s); |
|---|---|---|---|---|---|
| DS (%) | DS (%) | Entry | (mM) | (mM) | (% w/v) |
| 15 | 15 | 1 | 0.01 | 0.1 | 58 ± 5; (3.5) |
| | | 2 | 0.01 | 0.1 | 85 ± 5 (3.0) |
| | | 3 | 0.01 | 0.1 | 252 ± 5 (2.5) |
| 12 | 12 | 4 | 0.01 | 0.1 | 458 ± 20 (2.0) |
| | | 5 | 0.002 | 0.2 | 2400 ± 60 (2.0) |
| | | 6 | 0.02 | 0.5 | 23 ± 2 (2.0) |
| 10 | 10 | 7 | 0.002 | 0.5 | 30 ± 5 (2.0) |
| | | 8 | 0.002 | 0.02 | 55 ± 5 (2.0) |
| | | 9 | 0.001 | 0.02 | NG (2.0) |
| | | 10 | 0.0002 | 0.02 | NG (2.0) |
| | | 11 | 0.002 | 0.5 | 37 ± 2 (2.0) |

^{a}copper sulfate
^{b}sodium ascorbate
NG = no gelation was observed

### Swelling Study of Different Hydrogels as Reported in Example 11

Different gels were prepared using the conditions described in Table 8, entry 3 and 11. All swelling experiments were carried out 5 independent times. The swelling ratio was dependent on the ionic strength of the media. In water, the swelling ratio reaches its maximum (700%).

### Example 12

### Release Measurements of the Drug Benzydamine Hydrochloride from Material Based on Crosslinked Hyaluronic Acid Obtained in Example 11

The loading of the drug into polymer networks was carried out by swelling-equilibrium method and by physical incorporation. In the first procedure, the crosslinked polymers were allowed to swell in the drug solution of a known concentration for 24, 48 and 72 hours at 37° C. The materials were dried after that to obtain the drug-loaded device. The concentration of the rejected solution was measured to calculate the percent of entrapment in the polymer matrix. However, the absorption of the polymer network was very poor. The second tested methodology was the physical incorporation. For the release experiments, before gelation benzadymine solutions of known concentrations were prepared in phosphates saline-buffer, yielding a final concentration of 3, 4 and 5 g/L. 1 ml of this prepared solution was used for dissolving the components (Table 8, entry 11) with a molar ratio 1:1 which were hydrated overnight (total volume 1.0 ml). The exact amount of was determined based on their degree of substitution (DS) so that the final concentration of both reagents was kept at 2% (w/v) in case of DS=100%. The hydrogel slabs with a degree of crosslinking of 10% were prepared by click chemistry using a freshly prepared solution of copper sulfate and sodium ascorbate as the catalyst and initiator respectively (Table 8, entry 11). Gelation was allowed to proceed for two hours at room temperature before the kinetics determinations starts. The amount of the released drug was measured spectrophotometrically every 30 minutes in each case and for 2 days, when the sample has reached maximum swelling equilibrium. The cumulative release of benzydamine hydrochloride was calculated based on a calibration curve using a concentration which embraces the concentration of the incorporated drug before crosslinking.

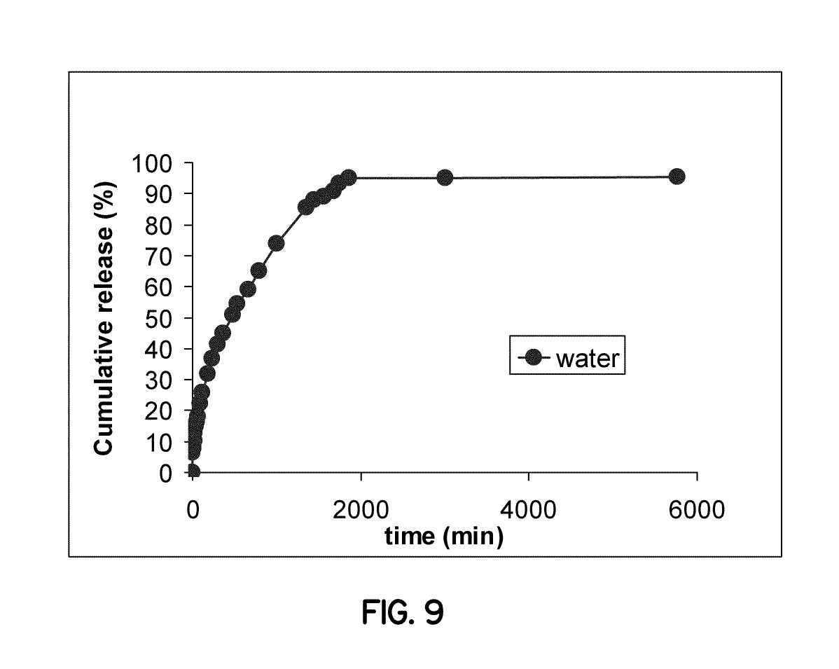

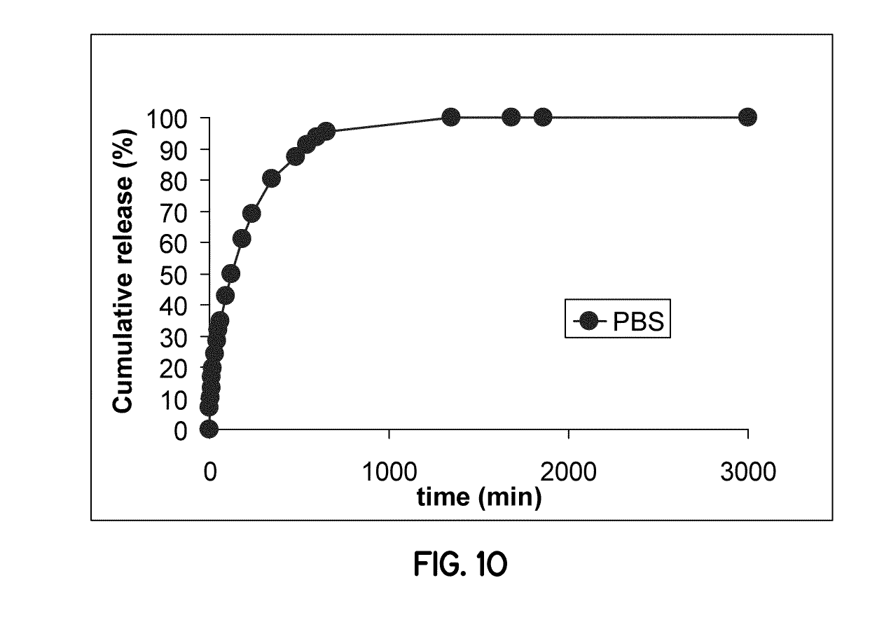

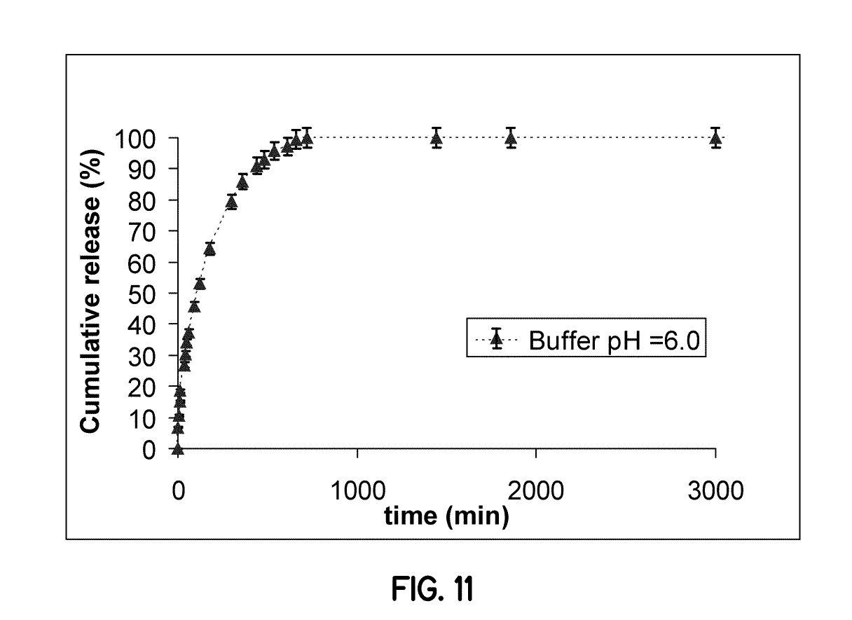

This example illustrates the kinetics of release of Benzydamine hydrochloride entrapped in the material which is freely released from the gels (drug release profiles were evaluated in vitro and are shown in FIGS. 9, 10 and 11). Higuchi and Ritger-Korsmeyer-Peppas equations (Korsmeyer, Gurny, Doelker, Buri & Peppas, 1983; Ritger & Peppas, 1987a, b) were used to analyze the effect of the components on the physical properties, which are model-independent methods. For model-dependent analysis, these two theoretical models fit the drug release from polymeric systems. Korsmeyer-Peppas model was used first to calculate the correlation coefficient for the obtained release data. Correlations of kinetic curves described by the model are presented in Table 9, using benzydamine hydrochloride as drug model for release into medium of different pH as resumed in Table 9.

TABLE 9
Korsmeyer-Peppas model used to evaluate the diffusion exponent (n).

| | | Diffusion | | | t₅₀ |
|---|---|---|---|---|---|
| entry | Effect of pH | Exponent (n) | Model | R² | time (min) |
| 1 | 2 | 0.3805 | K-Peppas | 0.9957 | 80 ± 10 |
| 2 | 4 | 0.4940 | K-Peppas | 0.9999 | 100 ± 10 |
| 3 | 6 | 0.5466 | K-Peppas | 0.9953 | 110 ± 10 |
| 4 | Distilled-water | 0.4840 | K-Peppas | 0.9997 | 480 ± 10 |
| 5 | 7.4 | 0.5323 | K-Peppas | 0.9996 | 120 ± 10 |
| 6 | 8 | 0.4707 | K-Peppas | 0.9998 | 95 ± 10 |
| 7 | 10 | 0.3389 | K-Peppas | 0.9995 | 7200 ± 20 |

t₅₀ represents the time when 50% of the initial incorporated drug is released
R² means the coefficient of determination after linear regression.

On the basis of the best fitted model parameters values, the dependence of the release rate is described. The amphoteric material described in this invention presents an anomalous diffusion mechanism; thus, the drug releases over time irrespective of the concentration and shows a clear dependence on pH of the release media. On the other hand, it was observed the n value has not significantly changed due to the concentration, which means that the transport mechanism is not changing due to the network inhomogenity. Using the Fickian model, which is commonly applied to determine apparent diffusion coefficients when intramolecular interactions are present, the results have shown that the apparent diffusion coefficient inside the hydrogels significantly differed from that in the solution. That difference is attributed to a hindered diffusion into the hydrogel as the guests follow a torturous diffusional path through the material. The transport mechanism for the materials reported in this patent follows Non-Fickian diffusion and not erosion, therefore the material can be considered stable during the analyzed time. The amount of the drug released in pH=2.0 buffer was lower in comparison to the release medium of pH=4.0, 6.0, 8.0 and 10.0. The 50% of the total release of drug at pH=2.0 occurred in 80 minutes±10, 100 minutes±10 minutes (4.0, 6.0, 8.0) and 7200 minutes±20 minutes for pH=10.0. The values of diffusion exponent and for the release are presented in Table 9.

Diffusion exponents (n) for pH=4, 6 and 8 suggest that the mechanism leading to the release of benzydamine had an anomalous transport with a constant release rate. This kind of mechanism is adequate for a controlled sustained release dosage form.

### Example 13

### Release Measurements of Doxorubicin Hydrochloride from Material BASED on Crosslinked Hyaluronic Acid Obtained in Example 10

For the release experiments, before gelation doxorubicine hydrochloride of a known concentration was prepared in phosphates saline-buffer, yielding a final concentration of 2 g/L. 1 ml of this prepared solution was used for dissolving the components in a molar ratio 1:1 which were hydrated overnight. The exact amount of was determined on the basis of their degree of substitution (DS) so that the final concentration of both reagents was kept at 2% (w/v) in case of DS=100%. The hydrogel slabs with a degree of crosslinking of 10% were prepared using conditions reported in Table 8, Entry 11.

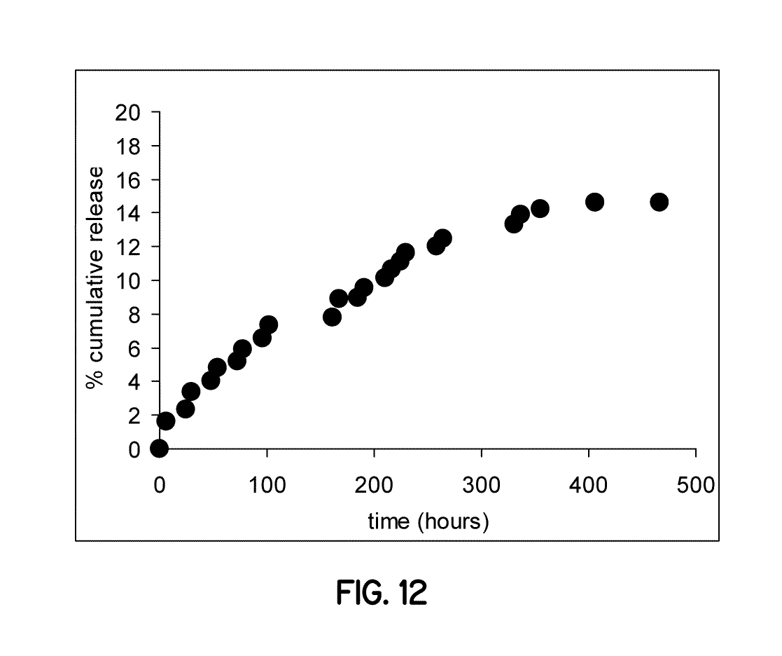

The amount of the released drug was measured spectrophotometrically every 10 hours in each case for 30 days. The cumulative release of doxorubicin was calculated based on a calibration curve using a concentration which embraces the incorporated amount of drug before the crosslinking, and is shown in FIG. 12. The maximum quantity of doxorubicin is released over a period of 466 h using a medium of saline-phosphate buffer and is equal to 15% of the initial quantity loaded in the hydrogel. Whereas using water as a release medium the maximum amount o doxorubicin released is again 15% but in a shorter period of time, in this case 186 h. In the case of doxorubicin the diffusion exponent indicates Fickian diffusion for the release in PBS medium and super case II transport system n>1. Similar trends have been observed for other kind of amphoteric hydrogels previously reported in literature (Ferrero, Muńoz-Ruiz & Jiménez-Castellanos, 2000; Rao, Devi & Buri, 1990). This type of mechanism can be attributed to an increase in plasticization at the relaxing boundary (gel layer) (Ritger & Peppas, 1987b). According to SEM Doxorubicin was not crystallized (Data not shown).

### Example 14

### Cell Viability, Proliferation and Differentiation on Chemically Modified Hyaluronan and Crosslinked Material

### Chemically Modified HA

Solutions of both derivatives of Table 8, entry 11, were prepared (1% w/v) in the case of DS=100%. In case of a modified polymer with the DS=10%, 10% of the polymer is modified to contain acetyl glucosamine part containing azide or propargyl attached group and 90% of the polymer has a non-modified acetyl glucosamine part. Therefore, out of every 10 dimers of hyaluronic acid, there are 9 non-modified dimers and 1 modified dimer. The exact amount of reagents is always determined on the basis of substitution degree or DS (determined by NMR). Therefore, the concentration of the reagent is calculated based on the modified part of HA but the concentration of the polymer always has to be expressed as % w/v of all the polymer because the modified part cannot be isolated from the whole macromolecule. The prepared solutions were filtrated to produce sterile solutions to be tested independently for viability and cytotoxicity. 2 000 (3T3) cells were seeded to wells of 96-well test plates. The cells were cultured for 24 hours before their treatment with the tested solutions. Then, the tested solution was added to each well so that the final concentration of the tested solution in the well is 100, 500, 1000 μg/mL using as diluent medium. Cell viability was measured 0, 24, 48, 72 hours after treatment using the 3-(4,5-dimethylthiazol-2-yl)-2,5-diphenyl tetrazolium bromide (MTT) assay. 20 μL of MTT stock solution (5 mg/ml) was added to 200 μL of cell culture medium in each well. In the assay, MTT is reduced by viable cells to a blue formazan salt, which is later released from the cells and determined spectrophotometrically. The plates were incubated at 37° C. in a cell culture incubator for 2.5 hours. Then all MTT solution was removed and 220 μl of lysis solution was added. Cells were lysed for 30 minutes at room temperature on a shaker and then the optical density was measured by Microplate reader VERSAmax at 570 nm according to a standard analytical protocol. The experiment design was completed with a set of control cells cultured in common media without treatment and blank samples. All experiments were carried out at least in six independent repeats.

Cell viability and attachment were assessed by fluorescence microscopy using a live/dead (Eukolight) viability kit. The porous structures observed for the crosslinked materials have suggest their potential use as scaffolds for cell infiltration and growth. Therefore, the viability of the crosslinked material was tested as well.

### Crosslinked Material Test

A sterile solution of the derivatives of degree of substitution DS=10% was prepared using PBS to obtain a final concentration of 2% (w/v) for a degree of substitution of 100%. The material was crosslinked by an addition of CuSO₄.5H₂O and sodium ascorbate (such an amount of CuSO₄.5H₂O so that the concentration thereof in the resulting solution is 0.002M and sodium ascorbate 0.02M). Cross-linked derivatives were washed out 3 times using 10 ml of PBS and 3 times using cell cultivation media. The material was completely mashed and homogenized. Then, the material was sterile lyophilized. 1.0 g of the mashed material (dry weight) was allowed to swell again using 8 ml of PBS. 0.5, 1.0, 1.3, 2.0 and 3.0 ml of this solution was added to each well (which already contains the cells) and the viability and cytotoxicity test was conducted in the same way as in case of the derivatives by direct contact with the cells. Viability test MTT was utilized in order to obtain the basic information about cell metabolism and proliferation for the “onto” the crosslinked material. This experiment was repeated three times. Optical density was measured and the percentages relative to the control were calculated as a ratio of optical density values to optical density value of untreated cells, multiplied 100 times on time for each repetition and then the mean was calculated.

Conclusion

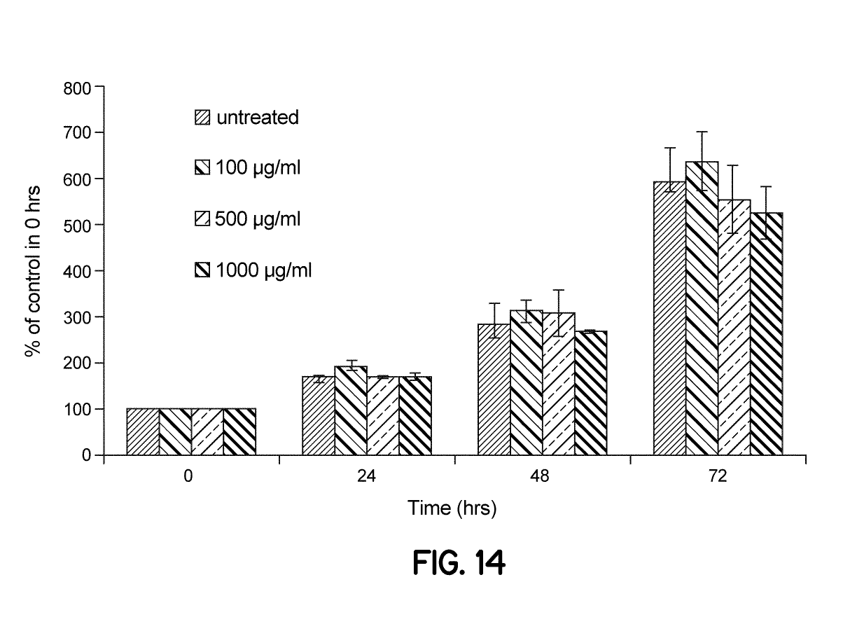

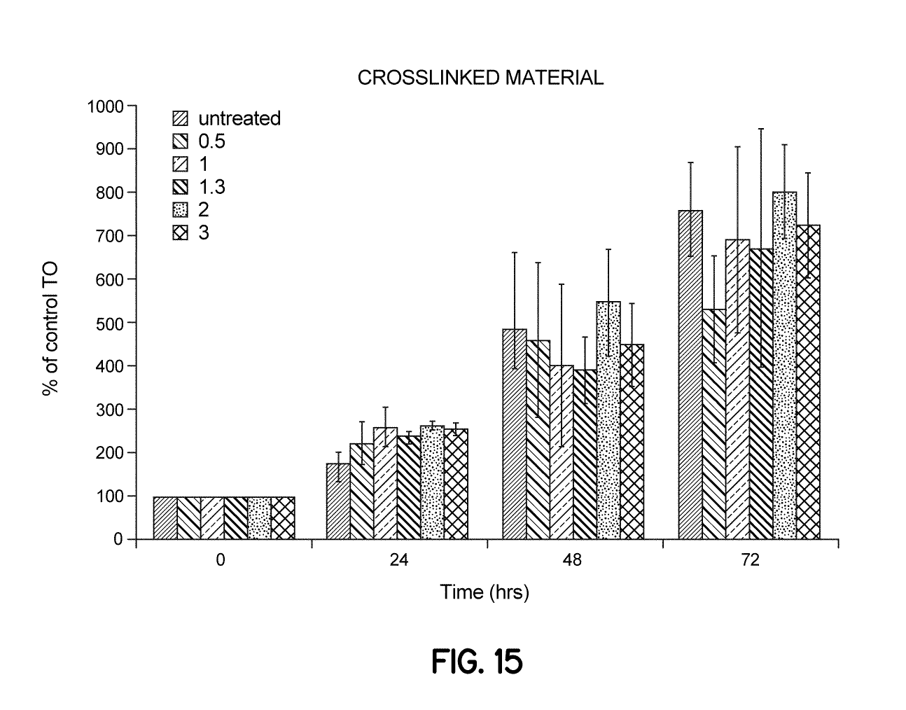

On the basis of the obtained results from cell viability, the derivatives disclosed in Table 8, entry 11 were not cytotoxic for cell line 3T3 in all the tested concentrations (100 μg/ml, 500 μm/ml, and 1000 μm/ml), The mean percentage relative to the control and standard error of the means (SEMs) were calculated and the effect of derivatives solution is shown in FIG. 13 for propargyl-2-amine-HA; and FIG. 14 for azidopropyl-2-amine-HA. Cross-linked derivatives were as well non-cytotoxic for cell line 3T3 (FIG. 15). The viability of cells after incubation in a medium containing different concentrations of derivatives as well as crosslinked derivatives had not changed significantly within the whole monitored interval: 24-72 hours after treatment.

### Example 15

### Chondrocytes Bio-Availability and Seeding in a Scaffold Based on II and III

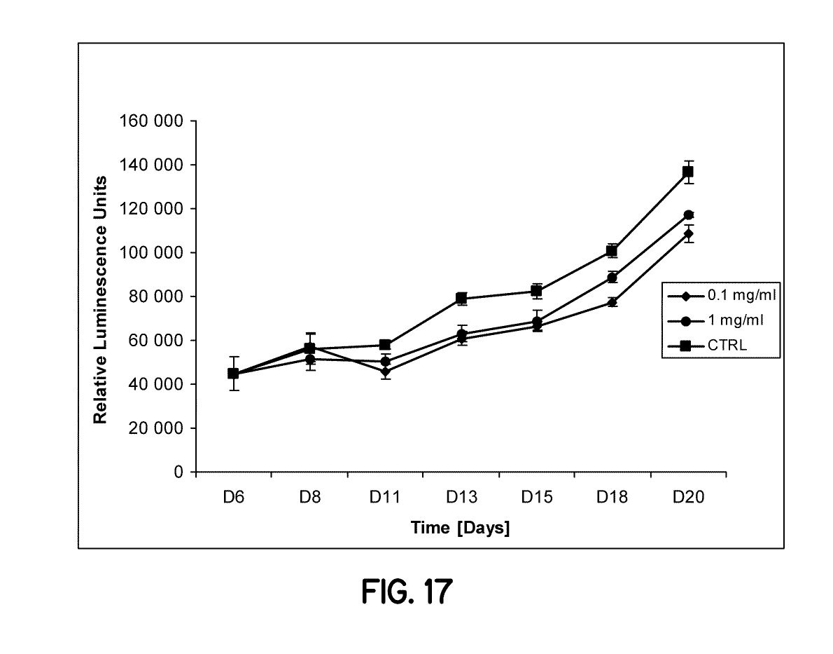

150 mg of HA-propargyl-2-amine and 150 mg of HA-azidopropyl-2-amine (both sterile) were dissolved in 3 ml PBS and mixed with 3 μl of 10 mM CuSO₄×5H₂O and 400 μl 2.5 M sodium ascorbate. This mixture was divided in 100 μl aliquots into 96-well test plate. Gelation took place in a thermobox at 37° C. for 2 hours. The newly formed gels were soaked into 1 mM Na₂EDTA (Sigma) bath and washed 3 times for 1 hour on orbital shaker. Then the gels were washed 1 hour in PBS buffer solution and next 4 times in injection water. Gels were frozen at −80° C. and lyophilized under sterile conditions. The gel seeding was effected by transfer of each lyophilized scaffold cylinder into a separate well of 24-well test plate and seeded with 200 μl of 3T3 cell suspension (1×10⁶ cells/ml) poured onto the top of scaffold. Suspension were let to stand for 1 hour to be absorbed into the dry scaffold in a thermobox at 37° C. and then the whole 24-well test plate with scaffolds were spun at 1200 rpm for 10 min. After centrifugation, 1 ml of the cultivation medium was added to each scaffold and placed in a thermobox at 37° C., 5% CO₂ and humidified atmosphere. Next day each scaffold was transferred to a new well with 1 ml of fresh medium. The medium was replaced every 2-3 days. Each scaffold was placed into 5 ml of PBS buffer solution with 4 μl calcein AM (Sigma) and 1 μl ethidium homodimer and placed into orbital shaker for 2.5 hour. Then each scaffold was washed 3 times with 20 ml PBS and observed by fluorescence microscope (FIG. 16a-d). FIG. 17 shows the viability test (ATP) of human chondrocytes (6P) in the presence of a scaffold made of crosslinked material based on the derivatives II and III, described in Scheme 7. For this testing two solutions were prepared using concentrations of 0.1 and 1% w/v, n=5, ±SEM. Cells were seeded in density of 5000 cells per cm² and pre-cultivated for 6 days. The crosslinked material has been tested after the 6th day and during 15 days of cultivation. This graph shows that the activity of the cells increased with the time which means that the cells had reproduced and grown into the scaffold.

Other embodiments are within the following claims. For example, the scale of the reactions may be increased for the commercial production of the composition of the invention. It will be also well understood by those skilled in the art that varying the ratio of the polyanionic polysaccharide will change the properties of the final material.

### REFERENCES USED FOR COMPARISON OF CHEMICAL PROPERTIES

Crescenzi, V., Cornelio, L., Di Meo, C., Nardecchia, S., & Lamanna, R. (2007). Novel Hydrogels via Click Chemistry: Synthesis and Potential Biomedical Applications. Biomacromolecules, 8(6), 1844-1850.
El-Sherbiny, I. M., & Smyth, H. D. C. Poly(ethylene glycol)-carboxymethyl chitosan-based pH-responsive hydrogels: photo-induced synthesis, characterization, swelling, and in vitro evaluation as potential drug carriers. Carbohydrate Research, 345(14), 2004-2012.
Ferrero, C., Muńoz-Ruiz, A., & Jiménez-Castellanos, M. R. (2000). Fronts movement as a useful tool for hydrophilic matrix release mechanism elucidation. International Journal of Pharmaceutics, 202(1-2), 21-28.
Ferruti, P., Bianchi, S., Ranucci, E., Chiellini, F., & Caruso, V. (2005). Novel Poly(amido-amine)-Based Hydrogels as Scaffolds for Tissue Engineering. Macromolecular Bioscience, 5(7), 613-622.
Gupta, P., Vermani, K., & Garg, S. (2002). Hydrogels: from controlled release to pH-responsive drug delivery. Drug Discovery Today, 7(10), 569-579.
Hasegawa, T., Umeda, M., Numata, M., Li, C., Bae, A.-H., Fujisawa, T., Haraguchi, S., Sakurai, K., & Shinkai, S. (2006). [‘]Click chemistry’ on polysaccharides: a convenient, general, and monitorable approach to develop (1-->3)-[beta]-d-glucans with various functional appendages. Carbohydrate Research, 341(1), 35-40.
Hoffman, A. S. (1995). “Intelligent” Polymers in Medicine and Biotechnology. Artificial Organs, 19(5), 458-467.
Chen, L., Tian, Z., & Du, Y. (2004). Synthesis and pH sensitivity of carboxymethyl chitosan-based polyampholyte hydrogels for protein carrier matrices. Biomaterials, 25(17), 3725-3732.
Kim, B., & Peppas, N. A. (2002). Complexation Phenomena in pH-Responsive Copolymer Networks with Pendent Saccharides. Macromolecules, 35(25), 9545-9550.
Korsmeyer, R. W., Gurny, R., Doelker, E., Buri, P., & Peppas, N. A. (1983). Mechanisms of solute release from porous hydrophilic polymers. International Journal of Pharmaceutics, 15(1), 25-35.
Luo, Y., Peng, H., Wu, J., Sun, J., & Wang, Y. Novel amphoteric pH-sensitive hydrogels derived from ethylenediaminetetraacetic dianhydride, butanediamine and amino-terminated poly(ethylene glycol): Design, synthesis and swelling behavior. European Polymer Journal, 47(1), 40-47.
Malkoch, M., Vestberg, R., Gupta, N., Mespouille, L., Dubois, P., Mason, A. F., Hedrick, J. L., Liao, Q., Frank, C. W., Kingsbury, K., & Hawker, C. J. (2006). Synthesis of well-defined hydrogel networks using Click chemistry. Chemical Communications (26), 2774-2776.
Pal, K., Paulson, A. T., Rousseau, D., Stefan, K., Ian, T. N., & Johan, B. U. (2009). Biopolymers in Controlled-Release Delivery Systems. Modern Biopolymer Science pp. 519-557). San Diego: Academic Press.

Qiu, Y., & Park, K. (2001). Environment-sensitive hydrogels for drug delivery. Advanced Drug Delivery Reviews, 53(3), 321-339.

Rao, K. V. R., Devi, K. P., & Buri, P. (1990). Influence of molecular size and water solubility of the solute on its release from swelling and erosion controlled polymeric matrices. Journal of Controlled Release, 12(2), 133-141.
Ritger, P. L., & Peppas, N. A. (1987a). A simple equation for description of solute release I. Fickian and non-fickian release from non-swellable devices in the form of slabs, spheres, cylinders or discs. Journal of Controlled Release, 5(1), 23-36.
Ritger, P. L., & Peppas, N. A. (1987b). A simple equation for description of solute release II. Fickian and anomalous release from swellable devices. Journal of Controlled Release, 5(1), 37-42.
Rostovtsev, V. V., Green, L. G., Fokin, V. V., & Sharpless, K. B. (2002). A Stepwise Huisgen Cycloaddition Process Copper(I)-Catalyzed Regioselective “Ligation” of Azides and Terminal Alkynes. Angewandte Chemie International Edition, 41(14), 2596-2599.
Shang, J., Shao, Z., & Chen, X. (2008). Chitosan-based electroactive hydrogel. Polymer, 49(25), 5520-5525.
Tankam, P. F., Müller, R., Mischnick, P., & Hopf, H. (2007). Alkynyl polysaccharides: synthesis of propargyl potato starch followed by subsequent derivatizations. Carbohydrate Research, 342(14), 2049-2060.
Testa, G., Di Meo, C., Nardecchia, S., Capitani, D., Mannina, L., Lamanna, R., Barbetta, A., & Dentini, M. (2009). Influence of dialkyne structure on the properties of new click-gels based on hyaluronic acid. International Journal of Pharmaceutics, 378(1-2), 86-92.
Tornøe, C. W., Christensen, C., & Meldal, M. (2002). Peptidotriazoles on Solid Phase: [1,2,3]-Triazoles by Regiospecific Copper(I)-Catalyzed 1,3-Dipolar Cycloadditions of Terminal Alkynes to Azides. Journal of Organic Chemistry, 67(9), 3057-3064.
van Bommel, K. J. C., van der Pol, C., Muizebelt, I., Friggeri, A., Heeres, A., Meetsma, A., Fering a, B. L., & van Esch, J. (2004). Responsive Cyclohexane-Based Low-Molecular-Weight Hydrogelators with Modular Architecture. Angewandte Chemie International Edition, 43(13), 1663-1667.
Yao, F., Chen, W., Liu, C., & De Yao, K. (2003). A novel amphoteric, pH-sensitive, biodegradable poly[chitosan-g-(L-lactic-co-citric) acid]hydrogel. Journal of Applied Polymer Science, 89(14), 3850-3854.

1. A crosslinked derivative of hyaluronic acid according to the formula I: 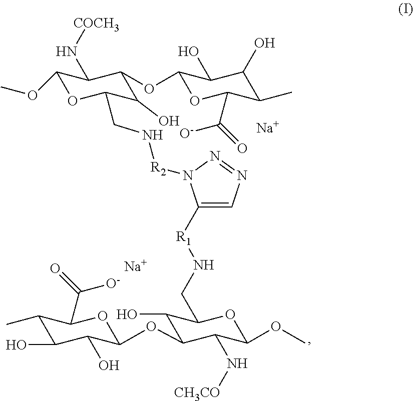
wherein R₁ and R₂ are independently the same or different and are selected from the group comprising an aliphatic, aromatic, arylaliphatic and cycloaliphatic moiety containing 3-12 carbons, optionally also containing a heteroatom O.

2. The crosslinked derivative according to claim 1, wherein R₁ is selected from the group comprising methyl and phenyl and R₂ is selected from the group comprising propyl, phenyl and 3,6,9-trioxaundecane.

3. The crosslinked derivative according to any of claims 1 to 2 possessing DS of 1 to 28%, preferably 10%, wherein DS is defined as the number of substituted dimers per 100 saccharide dimers.

4. A process of preparation of the derivative disclosed in any of claims 1 to 3, comprising the steps of
i) preparation of a hyaluronan derivative of the formula II carrying an alkynyl group bound via a secondary amine group, wherein R₁ is defined in claim 1: 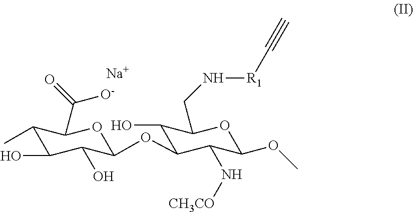
ii) preparation of a hyaluronan derivative of the formula III, carrying an azidyl group bound per a secondary amine group, wherein R₁ is defined in claim 1: 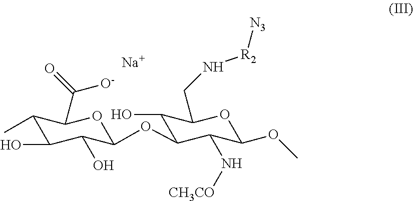
iii) mixing the derivative of the formula (II) and the derivative of the formula (III), and
iv) cycloaddition reaction of the derivative of the formula (II) and the derivative of the formula (III) in the presence of CuSO₄ and sodium ascorbate to obtain the crosslinked derivative of the hyaluronic acid of the general formula (I).

5. The process according to claim 4, wherein step i) comprises the steps of a) a chemoselective oxidation of hyaluronan in the C-6 position, b) coupling a primary amine carrying a terminal alkynyl group to the oxidized hyaluronan to form alkynyl-imine hyaluronan, c) reduction of the alkynyl-imine hyaluronan to form a secondary alkynyl-amine hyaluronan.

6. The process according to claim 5, wherein the step a) is followed by an isolation of the oxidized hyaluronan.

7. The process according to claim 5, wherein each of the steps a) to c) are performed in one pot.

8. The process according to any of claims 4-7, wherein step ii) comprises the steps of a) chemoselective oxidation of hyaluronan in the C-6 position, b) coupling a primary amine carrying a terminal azidyl group to the oxidized hyaluronan to form azidoalkyl-imine hyaluronan, c) reduction of the azidoalkyl-imine hyaluronan to form a secondary azidoalkyl amine hyaluronan.

9. The process according to claim 8, wherein the step a) is followed by an isolation of the oxidized hyaluronan.

10. The process according to claim 8, wherein each of the steps a) to c) are performed in one pot.

11. The process according to any of claims 5-10, wherein the primary amine carrying a terminal alkynyl group is selected from the group comprising propargyl amine and ethynyl aniline, and the primary amine carrying a terminal azidyl group is selected from the group comprising 3-azidopropan-amine, 11-azido-3,6,9-trioxaundecan-1-amine and azido-aniline.

12. The process according to any of claims 5-11, wherein the oxidation of hyaluronan in step a) is performed using the oxidation system 2,2,6,6,-tetramethylpiperidine-1-oxyl radical (TEMPO)/sodium hypochlorite (NaClO) and using NaX as an additive, where X═Br or Cl, or using Dess-Martin Periodinane (DMP).

13. The process according to any of claims 4-12, wherein it is carried out at a temperature range from 0 to 60° C., preferably from 5 to 37° C.

14. The process according to any of claims 4-13, wherein the crosslinked derivative obtained in step iv) is in the form of a gel which is then freeze-dried.

15. The process according to claim 14, wherein the freeze-drying is performed by liquid nitrogen or by ice.

16. The process according to any of claims 4-13, wherein step iii) further involves adding biologically active substances into the reaction mixture, said substances being selected from the group comprising drugs, proteins, enzymes, biopolymers and biologically compatible synthetic polymers.

17. The process according to claim 16, wherein the drugs are selected from the group comprising analgesics, antibiotics, antimicrobial agents, cytostatics, anticancer drugs, anti-inflammatory substances, wound healing agents and anesthetics.

18. The process according to any of claims 4-15, wherein step iv) is followed by seeding the formed crosslinked derivative with growth factors.

19. The process according to claim 18, wherein the growth factors are chondrocytes.

20. The crosslinked derivative of hyaluronic acid of the formula (I) according to any of claims 1 to 3 in the form of a gel or a scaffold.

21. The crosslinked derivative of hyaluronic acid of claim 20 further comprising entrapped biologically active substances selected from the group comprising drugs, proteins, growth factors, enzymes, biopolymers and biologically compatible synthetic polymers.

22. The crosslinked derivative of hyaluronic acid of claim 20 or 21, wherein it is in the form of a gel and it comprises a long-term release device.

23. Use of the crosslinked derivative according to the formula (I) for controlled release systems, in tissue engineering, wound dressing or tissue regeneration.
